(19) United States
(12) Patent Application Publication
(10) Pub. No.: US 2022/0105147 A1
(43) Pub. Date: Apr. 7, 2022

(54) CROTON LECHLERI COMPOSITIONS FOR USE IN THE TOPICAL TREATMENT OF ACUTE BACTERIAL SKIN OR SKIN STRUCTURE INFECTION

(71) Applicant: ALPHYN BIOLOGICS, LLC, Cincinnati, OH (US)

(72) Inventors: Gary Michael PEKOE, Cincinnati, OH (US); Jazmyne Kristyne MINK, Cincinnati, OH (US); Steven Aaron PENTELNIK, Cincinnati, OH (US); Neal G. KOLLER, Annapolis, MD (US)

(21) Appl. No.: 17/599,517

(22) PCT Filed: Mar. 20, 2020
(86) PCT No.: PCT/US2020/024067

§ 371 Date: Sep. 28, 2021

Related U.S. Application Data
(60) Provisional application No. 62/821,234, filed on Mar. 20, 2019.

(51) Int. Cl. A61K 36/47; A61K 31/353; A61K 9/00; A61P 31/04

(52) U.S. Cl. CPC A61K 36/47; A61P 31/04; A61K 9/0014; A61K 31/353

(57) ABSTRACT

The present disclosure provides for the treatment of acute bacterial skin or skin structure infections via the topical administration of a pharmaceutical composition comprising a therapeutically effective amount of an extract of the Croton lechleri tree. Also provided are details of studies on the effectiveness of an extract of the Croton lechleri tree on acute bacterial skin or skin structure infections and causative pathogens.

### CROSS-REFERENCE TO RELATED APPLICATIONS

This application claims the benefit of U.S. Provisional Application No. 62/821,234 filed Mar. 20, 2019. The disclosure of the application is incorporated herein by reference.

### SUMMARY

The present invention is generally related to the treatment of acute bacterial skin or skin structure infections (ABSSSI), uncomplicated or complicated, via the topical administration of a pharmaceutical compositions comprising a therapeutically effective amount of latex of Croton lechleri, preferably filtered latex of Croton lechleri, preferably filtered latex of Croton lechleri Müll.Arg.

### BRIEF DESCRIPTION OF THE DRAWINGS

FIG. 1 depicts a representative Total Ion Chromatogram as well as additional Multiple Reaction Monitoring spectra that identify the marker compounds in an AB-101 composition.

FIG. 2A depicts the NMR spectra of 3 lots of AB-101 in D₂O—the top spectra is for Lot 00, the middle spectra is for Lot 01, and the bottom spectra is for Lot 02.

FIG. 2B depicts the overlay of the NMR spectra of Lots 00, 01, and 02 of AB-101 in D₂O.

FIG. 3A depicts the Nuclear Magnetic Resonance (NMR) spectra of 3 lots of AB-101 in d₄-Methanol—the top spectra is for Lot 00, the middle spectra is for Lot 01, and the bottom spectra is for Lot 02.

FIG. 3B depicts the overlay of the NMR spectra of Lots 00, 01, and 02 of AB-101 in d₄-Methanol.

FIG. 4A depicts the NMR spectra of 4 lots of AB-101 in d₄-Methanol—the top spectra is for Lot 00, the upper middle spectra is for Lot 01, the lower middle is for Lot 02, and the bottom spectra is for Lot X.

FIG. 4B depicts the overlay of the NMR spectra of Lots 00, 01, 02, and X of AB-101 in d₄-Methanol.

FIG. 5 depicts bar graphs comparing the AB-101 lot analysis results for A) gallocatechin B) epigallocatechin C) catechin D) epicatechin and E) taspine.

FIG. 6 depicts the zone of inhibition of of methanol extracted AB-101 against methicillin-susceptible Staphylococcus aureus (MSSA) (on the left) and methicillin-resistant Staphylococcus aureus (MRSA) (on the right).

FIG. 7 depicts the MSSA recovered over time in time-kill kinetic assay.

FIG. 8 depicts the MRSA recovered over time in time-kill kinetic assay.

### DEFINITIONS

Before the present compositions and methods are described, it is to be understood that this invention is not limited to the particular processes, compositions, or methodologies described, as these may vary. It is also to be understood that the terminology used in the description is for the purpose of describing the particular versions or embodiments only and is not intended to limit the scope of embodiments herein which will be limited only by the appended claims. Unless specifically defined herein, all technical and scientific terms used herein have the same meanings as commonly understood by one of ordinary skill in the art. Although any methods and materials similar or equivalent to those described herein can be used in the practice or testing of embodiments of embodiments herein, the preferred methods, devices, and materials are now described. All publications mentioned herein are incorporated by reference in their entirety. Nothing herein is to be construed as an admission that embodiments herein are not entitled to antedate such disclosure by virtue of prior invention.

It must also be noted that as used herein and in the appended claims, the singular forms “a,” “an,” and “the” include plural reference unless the context clearly dictates otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean plus or minus 10% of the numerical value of the number with which it is being used. Therefore, about 50% means in the range of 45%-55%.

As used herein, the term “AB-101” maybe used interchagably with latex of Croton lechleri, preferably filtered latex of Croton lechleri, preferably filtered latex of Croton lechleri Müll.Arg. The latex is excreted material from the wounded trunk of Croton lechleri, preferably of Croton lechleri Müll.Arg.

As used herein, the term “acute bacterial skin or skin structure infection” is defined as a bacterial infection of the skin including but not limited to bacterial skin infections, drug resistant bacterial skin infections or multi drug resistant bacterial skin infections. Additionally, such infections may be uncomplicated or complicated, mild or serious. Such infections may be without a lesion, abscess or wound (e.g., primary infections, such as all forms of impetigo including but not limited to Mupirocin-resistant impetigo), or with a lesion, abscess or wound. Additionally, such infections may be of any size, including those with a any lesion 75 cm2 or larger (often referred to as Acute Bacterial Skin and Skin Structure Infections (or ABSSSIs)) or lesser sized skin infections (often referred to as Secondarily Infected Traumatic Lesions (or SITLs), Skin and Soft Tissue Infections (or SSTIs).

“Administering” when used in conjunction with a therapeutic, such as AB-101, means to administer a therapeutic directly into or onto a target tissue or to administer a therapeutic to a patient whereby the therapeutic positively impacts the tissue to which it is targeted. Thus, as used herein, the term “administering”, when used in conjunction with a composition of embodiments herein, can include, but is not limited to, providing the composition into or onto the target tissue; providing the composition to a patient by, e.g., topical application whereby the therapeutic reaches the target tissue. “Administering” a composition may be accomplished topically or in combination with other known techniques.

As used herein the term “cellulitis/erysipelas” is defined as a diffuse bacterial skin infection characterized by spreading areas of redness, edema, and/or induration.

The transitional term “comprising,” which is synonymous with “including,” “containing,” or “characterized by,” is inclusive or open-ended and does not exclude additional, unrecited elements or method steps.

In embodiments or claims where the term “comprising” is used as the transition phrase, such embodiments can also be envisioned with replacement of the term “comprising” with the terms “consisting of” or “consisting essentially of.”

As used herein, the term “consists of” or “consisting of” means that the pharmaceutical composition, composition or the method includes only the elements, steps, or ingredients specifically recited in the particular claimed embodiment or claim.

As used herein, the term “consisting essentially of” or “consists essentially of” means that the pharmaceutical composition, or the method includes only the elements, steps or ingredients specifically recited in the particular claimed embodiment or claim and may optionally include additional elements, steps or ingredients that do not materially affect the basic and novel characteristics of the particular embodiment or claim. For example, the only active ingredient(s) in the composition or method that treats the specified condition (e.g., nutrient depletion) is the specifically recited therapeutic(s) in the particular embodiment or claim.

The term “combination therapy” means the administration of two or more therapeutic agents to treat a condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner In either case, the treatment regimen will provide beneficial effects of the drug combination in treating the conditions or disorders described herein.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “excipient” and “pharmaceutically acceptable excipient” as used herein are intended to be generally synonymous, and is used interchangeably with, the terms “carrier,” “pharmaceutically acceptable carrier,” “diluent,” “pharmaceutically acceptable diluent.”

As used herein the term “major cutaneous abscess” is defined as a bacterial infection characterized by a collection of pus within the dermis or deeper that is accompanied by redness, edema, and/or induration.

The term “patient” is generally synonymous with the term “subject” and includes all mammals including humans Examples of patients include humans, livestock such as cows, goats, sheep, pigs, and rabbits, and companion animals such as dogs, cats, rabbits, and horses. Preferably, the patient is a human.

As used herein, the term “pharmaceutically acceptable salt” refers to a salt prepared from a base or acid which is acceptable for administration to a patient, such as a mammal. The term “pharmaceutically acceptable salts” embraces salts commonly used to form alkali metal salts and to form addition salts of free acids or free bases. The nature of the salt is not critical, provided that it is pharmaceutically-acceptable. Such salts can be derived from pharmaceutically-acceptable inorganic or organic bases and from pharmaceutically-acceptable inorganic or organic acids.

As used herein, the term “therapeutic” or “therapeutic agent” or “pharmaceutically active agent” means an agent utilized to treat, combat, ameliorate, prevent or improve an unwanted condition or disease of a patient. In part, embodiments of the present invention are directed to the treatment of acute bacterial skin or skin structure infection, including, but not limited to Streptococcus pyogenes infection, Staphylococcus aureus infection, methicillin-resistant Staphylococcus aureus (MRSA) infection, Mupirocin-resistant MRSA, Enterococcus faecalis infection, Gram-positive bacteria infection, Gram-negative bacteria infection, cellulitis/erysipelas, wound infection, burn infection, major cutaneous abscesses, impetigo, Mupirocin-resistant impetigo, Vancomycin resistant bacteria infection, Mupirocin resistant bacteria infection, Clostridium difficile infection, drug-resistant Neisseria gonorrhoeae infection, Streptococcus pneumoniae infection, drug-resistant Streptococcus pneumoniae infection, drug-resistant Klebsiella pneumoniae infection, drug-resistant Malaria infection, Multi-drug resistant (MDR) infection, Extensively drug-resistant (XDR) Tuberculosis infection, Escherichia coli (E. coli) infection, Shiga toxin-producing Escherichia coli (E. coli) infection, infections caused by bacteria possessing Enzyme NDM-1 (New Delhi Metallo-beta-lactamase-1), Clostridium difficile infection, Enterococcus infection, Mycobacterium tuberculosis infection, Mycoplasma genitalium infection, Streptococcus infection, Campylobacter infection, Neisseria gonorrhoeae infection, Gamma proteobacteria infection, Enterobacteriaceae infection, Carbapenem-Resistant Enterobacteriaceae, infection, Klebsiella pneumoniae infection, Salmonella infection, E. coli infection, Pseudomonadales infection, Acinetobacter infection, Pseudomonas aeruginosa infection, MDR Pseudomonas aeruginosa infection, and Coagulase-negative Staphylococcus infection.

The term “therapeutically acceptable” refers to those compositions which are suitable for use in contact with the tissues of patients without undue toxicity, irritation, and allergic response, are commensurate with a reasonable benefit/risk ratio, and are effective for their intended use.

The term “therapeutically acceptable salt,” as used herein, represents salts or zwitterionic forms of the compositions disclosed herein which are water or oil-soluble or dispersible and therapeutically acceptable as defined herein. The salts can be prepared during the final isolation and purification of the compositions or separately by reacting the appropriate composition in the form of the free base with a suitable acid.

The phrase “therapeutically effective” is intended to qualify the amount of active ingredients used in the treatment of a disease or disorder or on the effecting of a clinical endpoint.

A “therapeutically effective amount” or “effective amount” of a composition is a predetermined amount calculated to achieve the desired effect, i.e., but not limited to to, inhibit, block, or reverse the activation, migration, or proliferation of cells. The activity contemplated by the present methods includes both medical therapeutic and/or prophylactic treatment, as appropriate. The specific dose of a composition administered according to this invention to obtain therapeutic and/or prophylactic effects will, of course, be determined by the particular circumstances surrounding the case, including, for example, the composition administered, the route of administration, and the condition being treated. The compositions are effective over a wide dosage range and, for example, dosages per application will normally fall within the range of from 0.001 to 10 mg/kg, more usually in the range of from 0.01 to 1 mg/kg. However, it will be understood that the effective amount administered will be determined by the physician in the light of the relevant circumstances including the condition to be treated, the choice of composition to be administered, and the chosen route of administration, and therefore the above dosage ranges are not intended to limit the scope of the invention in any way. A therapeutically effective amount of the composition of this invention is typically an amount such that when it is administered in a physiologically tolerable excipient composition, it is sufficient to achieve an effective systemic concentration or local concentration in the tissue.

The terms “treat,” “treated,” “treating”, or “treatment” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “topical” includes administering to any skin or mucosal surface or being suitable for such administration. In some embodiments, “topical” may be the skin surface. Skin surface includes any part of the body, including but not limited to face, hands, legs, neck, abdominal area, eyes, nose, and chest. Mucosal surface includes, without limitation, mucosa of the mouth or oral mucosa, lips, tongue, nasal, buccal mucosa, palate, gingiva, nasopharynx, respiratory epithelium, conjunctiva, vagina, cervix, and urethral mucosa.

As used herein the term “wound infection” is defined as a bacterial infection that may include purulent drainage from a wound with surrounding redness, edema, pain, tenderness and/or induration.

Also provided are embodiments wherein any embodiment herein may be combined with any one or more of the other embodiments, unless otherwise stated and provided the combination is not mutually exclusive.

Croton lechleri (a member of the family Euphorbiaceae, commonly called the spurge family) has approximately 1,300 species of plants that are either herbaceous (plants that have no persistent woody stem above ground), shrub (a woody plant which is smaller than a tree and has several main stems arising at or near the ground), tree (a perennial plant with an elongated stem, or trunk, supporting branches and leaves in most species), or liana (any of various long-stemmed, woody vines that are rooted in the soil at ground level and use trees, as well as other means of vertical support, to climb up to the canopy to get access to well-lit areas of the forest) forms. The Croton genus is a diverse and complex group of flowering plants ranging from herbs and shrubs to trees. The Croton genus is widely distributed in tropical and subtropical regions around the world.

Dragon's blood refers to a bright red resin that is obtained from different species of a number of distinct plant genus: Croton, Dracaena, Daemonorops, Calamus rotang and Pterocarpus. The red resin has been in continuous use since ancient times as varnish, medicine, incense, and dye. The name dragon's blood is used to refer to all of the above plant genus, often without any distinction as to the genus or species it is coming from. Those with the same genus will be similar in any therapeutic or nutritional value, with factors such as local soil, local rainfall, local humidity, local sunlight, local fauna and the like imparting variability and inconsistency. However, the difference between the red resin coming from Croton versus Daemonorops (a genus of rattan palms in the family Arecaceae found primarily in the tropics and subtropics of southeastern Asia with a few species extending into southern China and the Himalayas) will be significant. The Croton and Daemonorops genus originate from opposite sides of the world so their components are different and therefore specificity of source plant is important to deliver the desired medicinal benefits or avoid undesirable toxic results. For example milky white latex that is often toxic or at least irritating to the skin is common to the members of the spurge or Euphorbiaceae family Therefore selecting the specific genus, species, and local geographical area of the spurge or Euphorbiaceae family is essential to having the possiblity for the latex to have specific and repetitive medicinal properties.

A handful of Croton species found in the South America rainforest (in countries of Bolivia, Brazil, Colombia, Ecuador and Peru) Central America and Mexico produce the red latex, commonly known as dragon's blood, that has medicinal properties. The dragon's blood trees grown in these areas include Croton lechleri, Croton draco, Croton palanostigma, Croton sordidus, Croton urucurana, and Croton xalapenesis.

While the desired medicinal properties could be found by extracting the compositions from either the leaves or bark, in preferred embodiments, it is the deep red latex of Croton lechleri, preferably filtered latex of Croton lechleri, preferably filtered latex of Croton lechleri Müll.Arg, that is also referred to as latex, that is utilized. According to Langenheim (2003) resin “is a lipid-soluble mixture of volatile and non-volatile terpenoid and/or phenolic secondary compounds that are usually secreted in specialized structures located either internally or on the surface of the plant and are of potential significance in ecological interactions”. By contrast, latex, is a mixture of terpenoids, phenolic compounds, acids, carbohydrates, etc. having a protective role (Lewisohn 1991) and produced in special cells called laticifers (Fahn 1979). Chemical characterization of dragon's blood is species specific and has been undertaken by many authors. For example, it is possible to distinguish between dragon's blood from some individual species used in works of art, since it has been sold as a colorant for many centuries (Baumer and Dietemann 2010). Dragon's blood of Croton spp. is usually referred to as latex due to the fact that it is secreted and stored by laticifers, and its major constituents are polymeric anthocyanidins, which co-occur with many minor constituents, including diterpenes and simple phenols (Salatino et al. 2007). Dragon's blood secreted by stems of Pterocarpus officinalis is also called latex (Weaver 1997; Guerrero and Guzman 2004); however, information about the chemical composition of the exudate and its ecological function is poorly known. Dragon's blood derived from species of Dracaena and Daemonorops is a phenolic resin (Langenheim 2003), with well-recognized chemical content (e.g. Gonzalez et al. 2000; Shen et al. 2007; Sousa et al. 2008). Sometimes, dragon's blood is referred to as latex (e.g. Philipson 2001). However, this could prove to be a source of confusion, since plants produce other exudates referred to by that name, such as xylem latex and phloem latex, which are entirely different in terms of their location, chemical composition and function. The resin is obtained through tapping the tree or other common draining methods. Draining the tree latex has the additional benefit of not having to use complex and costly extraction technology to obtain the desired composition from either the leaves or bark. The latex of Croton lechleri Müll.Arg. of the present application is then filtered in a 30 micron filter to remove plant debris and thick, resinous material. Chemical characterization of dragon's blood is local geography specific and has not been undertaken by prior authors.

Medicinal and toxic properties of various species of the Croton genus have been ascribed to a wide variety of chemical compounds, such as terpenoids and steroids, alkaloids, and phenolic compounds, the latter including predominantly flavonoids, lignans, and proanthocyanidins. Some embodiments of the present application utilize the whole latex, thereby leveraging the “organic” synergy of all the latex components as intended by nature. The molecular classes found in latex of Croton lechleri Müll.Arg. of the present application which provide the desired medicinal benefits of Croton lechleri Müll.Arg. are: Alkaloids, Diterpenes, Lignans, Phenols, Phytosterols, Proanthocyanidins, Sterols and Tannins.

In certain embodiments, the specific dragon's blood tree of the present application is Croton lechleri Müll.Arg. of the Family: Euphorbiaceae. Dragon's blood is also referred to as Sangre de drago (Peru), Sangre de grado (Ecuador). Embodiments of the present invention are directed to pharmaceutical compositions of latex of Croton lechleri, preferably filtered latex of Croton lechleri, preferably filtered latex of Croton lechleri Müll.Arg and a pharmaceutically acceptable excipient. Such pharmaceutical compositions have been found to be useful in the successful treatment of acute bacterial skin or skin structure infections using the same. In some embodiments the pharmaceutical compositions are administered topically. Embodiments are directed to pharmaceutical compositions comprising latex of Croton lechleri, preferably filtered latex of Croton lechleri, preferably filtered latex of Croton lechleri Müll.Arg disclosed herein together with a pharmaceutically acceptable carrier, as well as methods of making and using the compositions. Certain embodiments are directed to methods for inhibiting acute bacterial skin or skin structure infections. Other embodiments are directed to methods for treating acute bacterial skin or skin structure infections in a patient in need of such treatment, comprising administering to said patient a therapeutically effective amount of a composition according to the present invention. Also provided is the use of certain extracts of Croton lechleri disclosed herein in the manufacture of a medicament for the treatment of acute bacterial skin or skin structure infections.

### Pharmaceutical Compositions

Embodiments herein are directed to pharmaceutical compositions comprising latex of Croton lechleri, preferably filtered latex of Croton lechleri, preferably filtered latex of Croton lechleri Müll.Arg., wherein the pharmaceutical composition does not contain a pharmaceutically acceptable excipient. In certain embodiments, latex of Croton lechleri, preferably filtered latex of Croton lechleri, preferably filtered latex of Croton lechleri Müll.Arg. comprises one or more compounds selected from: gallocatechin, epigallocatechin, catechin, epicatechin, and taspine, and combinations thereof. Each of gallocatechin, epigallocatechin, catechin, epicatechin, and taspine may be present in the latex of Croton lechleri, preferably filtered latex of Croton lechleri, preferably filtered latex of Croton lechleri Müll.Arg. in at least the amounts found in Table 1 or any combination of such amounts.

Embodiments herein are directed to pharmaceutical compositions comprising latex of Croton lechleri, preferably filtered latex of Croton lechleri, preferably filtered latex of Croton lechleri Müll.Arg. and a pharmaceutically acceptable excipient. In certain embodiments, latex of Croton lechleri, preferably filtered latex of Croton lechleri, preferably filtered latex of Croton lechleri Müll.Arg. comprises one or more compounds selected from: gallocatechin, epigallocatechin, catechin, epicatechin, and taspine, and combinations thereof. Each of gallocatechin, epigallocatechin, catechin, epicatechin, and taspine may be present in the latex of Croton lechleri, preferably filtered latex of Croton lechleri, preferably filtered latex of Croton lechleri Müll.Arg. in at least the amounts found in Table 1 or any combination of such amounts.

TABLE 1

| | Exemplary Amount present |
|---|---|
| Compound | in the latex (PPM is in μg/g) |
| Gallocatechin | at least about 110 PPM |
| Epigallocatechin | at least about 780 PPM |
| Catechin | at least about 1.6 PPM |
| Epicatechin | at least about 2 PPM |
| Taspine | at least about 45 PPM |

If the latex of Croton lechleri, preferably filtered latex of Croton lechleri, preferably filtered latex of Croton lechleri Müll.Arg. fails to contain the amounts of gallocatechin, epigallocatechin, catechin, epicatechin, and taspine in at least the amounts set forth in Table 1, it is not suitable for use in the pharmaceutical compositions and methods of use described herein.

In some embodiments, the gallocatechin is in an amount of at least about 110 mg, at least about 115 mg, at least about 120 mg, at least about 125 mg, at least about 130 mg, at least about 135 mg, at least about 140 mg, at least about 145 mg, at least about 150 mg, at least about 155 mg, at least about 160 mg, at least about 165 mg, at least about 170 mg, at least about 175 mg, at least about 180 mg, at least about 185 mg, at least about 190 mg, at least about 195 mg, at least about 200 mg, or a range between any two of these values.

In some embodiments, the epigallocatechin is in an amount of at least about 780 mg, at least about 790 mg, at least about 800 mg, at least about 810 mg, at least about 820 mg, at least about 830 mg, at least about 840 mg, at least about 850 mg, at least about 860 mg, at least about 870 mg, at least about 880 mg, at least about 890 mg, at least about 900 mg, at least about 910 mg, at least about 920 mg, at least about 930 mg, at least about 940 mg, at least about 950 mg, at least about 960 mg, at least about 970 mg, at least about 980 mg, at least about 990 mg, at least about 1000 mg, at least about 1010 mg, at least about 1020 mg, at least about 1030 mg, at least about 1040 mg, at least about 1050 mg, at least about 1060 mg, at least about 1070 mg, at least about 1080 mg, at least about 1090 mg, at least about 1100 mg, at least about 1110 mg, at least about 1120 mg, at least about 1130 mg, at least about 1140 mg, at least about 1150 mg, at least about 1160 mg, at least about 1170 mg, at least about 1180 mg, at least about 1190 mg, at least about 1200 mg, at least about 1210 mg, at least about 1220 mg, at least about 1230 mg, at least about 1240 mg, at least about 1250 mg, at least about 1260 mg, at least about 1270 mg, at least about 1280 mg, at least about 1290 mg, at least about 1300 mg, at least about 1310 mg, at least about 1320 mg, at least about 1330 mg, at least about 1340 mg, at least about 1350 mg, at least about 1360 mg, at least about 1370 mg, at least about 1380 mg, at least about 1390 mg, at least about 1400 mg, at least about 1410 mg, at least about 1420 mg, at least about 1430 mg, at least about 1440 mg, at least about 1450 mg, at least about 1460 mg, at least about 1470 mg, at least about 1480 mg, at least about 1490 mg, at least about 1500 mg, at least about 1510 mg, at least about 1520 mg, at least about 1530 mg, at least about 1540 mg, at least about 1550 mg, at least about 1560 mg, at least about 1570 mg, at least about 1580 mg, at least about 1590 mg, at least about 1600 mg, at least about 1610 mg, at least about 1620 mg, at least about 1630 mg, at least about 1640 mg, at least about 1650 mg, at least about 1660 mg, at least about 1670 mg, at least about 1680 mg, at least about 1690 mg, at least about 1700 mg, or a range between any two of these values.

In some embodiments, the catechin is in an amount of at least about 1.6 mg, at least about 1.7 mg, at least about 1.8 mg, at least about 1.9 mg, at least about 2.0 mg, at least about 2.1 mg, at least about 2.2 mg, at least about 2.3 mg, at least about 2.4 mg, at least about 2.5 mg, at least about 2.6 mg, at least about 2.7 mg, at least about 2.8 mg, at least about 2.9 mg, at least about 3.0 mg, at least about 3.1 mg, at least about 3.2 mg, at least about 3.3 mg, at least about 3.4 mg, at least about 3.5 mg, at least about 3.6 mg, at least about 3.7 mg, at least about 3.8 mg, at least about 3.9 mg, at least about 4.0 mg, at least about 4.1 mg, at least about 4.2 mg, at least about 4.3 mg, at least about 4.4 mg, at least about 4.5 mg, at least about 4.6 mg, at least about 4.7 mg, at least about 4.8 mg, at least about 4.9 mg, at least about 5.0 mg, at least about 5.1 mg, at least about 5.2 mg, at least about 5.3 mg, at least about 5.4 mg, at least about 5.5 mg, at least about 5.6 mg, at least about 5.7 mg, at least about 5.8 mg, at least about 5.9 mg, at least about 6.0 mg, at least about 6.1 mg, at least about 6.2 mg, at least about 6.3 mg, at least about 6.4 mg, at least about 6.5 mg, at least about 6.6 mg, at least about 6.7 mg, at least about 6.8 mg, at least about 6.9 mg, at least about 7.0 mg, at least about 7.1 mg, at least about 7.2 mg, at least about 7.3 mg, at least about 7.4 mg, at least about 7.5 mg, at least about 7.6 mg, at least about 7.7 mg, at least about 7.8 mg, at least about 7.9 mg, at least about 8.0 mg, at least about 8.1 mg, at least about 8.2 mg, at least about 8.3 mg, at least about 8.4 mg, at least about 8.5 mg, at least about 8.6 mg, at least about 8.7 mg, at least about 8.8 mg, at least about 8.9 mg, at least about 9.0 mg, at least about 9.1 mg, at least about 9.2 mg, at least about 9.3 mg, at least about 9.4 mg, at least about 9.5 mg, at least about 9.6 mg, at least about 9.7 mg, at least about 9.8 mg, at least about 9.9 mg, at least about 10.0 mg, at least about 10.1 mg, at least about 10.2 mg, at least about 10.3 mg, at least about 10.4 mg, at least about 10.5 mg, at least about 10.6 mg, at least about 10.7 mg, at least about 10.8 mg, at least about 10.9 mg, at least about 11.0 mg, or a range between any two of these values.

In some embodiments, the epicatechin is in an amount of at least about 2.0 mg, at least about 2.1 mg, at least about 2.2 mg, at least about 2.3 mg, at least about 2.4 mg, at least about 2.5 mg, at least about 2.6 mg, at least about 2.7 mg, at least about 2.8 mg, at least about 2.9 mg, at least about 3.0 mg, at least about 3.1 mg, at least about 3.2 mg, at least about 3.3 mg, at least about 3.4 mg, at least about 3.5 mg, at least about 3.6 mg, at least about 3.7 mg, at least about 3.8 mg, at least about 3.9 mg, at least about 4.0 mg, at least about 4.1 mg, at least about 4.2 mg, at least about 4.3 mg, at least about 4.4 mg, at least about 4.5 mg, at least about 4.6 mg, at least about 4.7 mg, at least about 4.8 mg, at least about 4.9 mg, at least about 5.0 mg, at least about 5.1 mg, at least about 5.2 mg, at least about 5.3 mg, at least about 5.4 mg, at least about 5.5 mg, at least about 5.6 mg, at least about 5.7 mg, at least about 5.8 mg, at least about 5.9 mg, at least about 6.0 mg, at least about 6.1 mg, at least about 6.2 mg, at least about 6.3 mg, at least about 6.4 mg, at least about 6.5 mg, at least about 6.6 mg, at least about 6.7 mg, at least about 6.8 mg, at least about 6.9 mg, at least about 7.0 mg, at least about 7.1 mg, at least about 7.2 mg, at least about 7.3 mg, at least about 7.4 mg, at least about 7.5 mg, at least about 7.6 mg, at least about 7.7 mg, at least about 7.8 mg, at least about 7.9 mg, at least about 8.0 mg, at least about 8.1 mg, at least about 8.2 mg, at least about 8.3 mg, at least about 8.4 mg, at least about 8.5 mg, at least about 8.6 mg, at least about 8.7 mg, at least about 8.8 mg, at least about 8.9 mg, at least about 9.0 mg, at least about 9.1 mg, at least about 9.2 mg, at least about 9.3 mg, at least about 9.4 mg, at least about 9.5 mg, at least about 9.6 mg, at least about 9.7 mg, at least about 9.8 mg, at least about 9.9 mg, at least about 10.0 mg, or a range between any two of these values.

In some embodiments, the taspine is in an amount of 45 mg, at least about 46 mg, at least about 47 mg, at least about 48 mg, at least about 49 mg, 50 mg, at least about 51 mg, at least about 52 mg, at least about 53 mg, at least about 54 mg, at least about 55 mg, at least about 56 mg, at least about 57 mg, at least about 58 mg, at least about 59 mg, at least about 60 mg, at least about 61 mg, at least about 62 mg, at least about 63 mg, at least about 64 mg, at least about 65 mg, or a range between any two of these values.

The pharmaceutical composition of AB-101 as described and claimed herein is a plant sourced material that meets the criteria of being consistently reproducible between batch to batch and reliably delivers the desired health benefits via topical application that may be used in a pharmaceutical composition. It can be used to treat Acute Bacterial Skin or Skin Structure Infections. Plant sourced materials face the challenge that changes in environmental weather, climate, rainfall, time of harvest (via season, time of day or month), changes in geography, longitude location, latitude location, altitude, changes in soil condition, harvesting protocols and many additional conditions can alter the characteristics of the plant that could impact quality. This can impact the plant's bioactivity resulting in inconsistency in achieving desired performance outcome. This creates a challenge in defining a pharmaceutical grade of dragon's blood to deliver consistent and reproducible therapeutic benefits. This is further compounded by the wide variety of the different species called dragon's blood. For example, phytochemical and anti-staphylococcal biofilm assessment of Dracaena draco L. Spp .draco resin, referred as dragon's blood, is “inactive in the maximum tested concentration of 1000 mcg/ml against free living staphylococci.” In contrast, AB-101 (latex of Croton lechleri, preferably filtered latex of Croton lechleri, preferably filtered latex of Croton lechleri Müll.Arg. with the appropriate levels of gallocatechin, epigallocatechin, catechin, epicatechin, and taspine) is effective against Staphylococcus specifically methicillin-susceptible Staphylococcus aureus (MSSA) or the shorten nomenclature staph bacteria and in particular methicillin-resistant Staphylococcus aureus (MRSA) and in particular Mupirocin resistant MRSA. The generic name of the Croton lechleri resin, ie, dragon's blood, or Sangre de grado, creates confusion in defining a plant-derived pharmaceutical and demonstrates that not all Croton lechleri plants are the same, nor do they provide similar benefits.

The benefits of AB-101, filtered or unfiltered latex of Croton lechleri, preferably filtered latex of Croton lechleri, preferably filtered latex of Croton lechleri Müll.Arg., is it's ability to deliver consistent results for treating the pathogens between batch to batch in spite of all the confounding conditions. The challenge in using the whole latex is to identify the compounds that deliver performance based on the many bio-active compounds comprising the latex. Even within the same species, grown in a similar location, there are variations in chemical content and bioactivity of the whole latex that unexpectedly varies in its ability to fight and kill pathogens.

Methodology that can identify the whole latex is effective by having an assay that determines when a batch meets the predetermined performance criteria. Having a unique analytical and microbiological assay enables the ability to identify which batch of filtered or unfiltered latex of Croton lechleri, preferably filtered latex of Croton lechleri, preferably filtered latex of Croton lechleri Müll.Arg, has the combination of components that will consistently deliver the desired outcome.

AB-101 botanical raw material (BRM) is a complex botanical product that is a latex of Croton lechleri, preferably filtered latex of Croton lechleri, preferably filtered latex of Croton lechleri Müll.Arg. that contains certain marker compounds (catechin, gallocatechin, epicatechin, epigallocatechin, and taspine) in specified amounts (see Table 1). Utilization of liquid chromatography with tandem mass spectrometry (LC-MS/MS) can be used to characterize the existence and levels of such marker compounds for batch to batch consistency and repeatable performance of AB-101. Marker compounds in AB-101 BRM include the proanthocyanidins: catechin, gallocatechin, epicatechin, and epigallocatechin, as well as the alkaloid taspine.

The published and accepted taxonomic classification of Croton lechleri Müll.Arg. is the following (van Ee & Berry, 2011, Riina et al, 2009, The Plant List, 2012, The Angiosperm Phylogeny Group, 2009):
Division: Streptophyta
   Class: Equisetopsida
      Subclass: Magnoliidae
         Order: Malpighiales
            Family: Euphorbiaceae
            Genus: Croton
            Subgenus Adenophylli
            Section: Cyclostigma
            Subsection: Cyclostigma

Species: Croton lechleri Müll.Arg.

Biodiversity of botanicals plays a major role in constituent chemical compound characterization. Chemical compounds utilized for as important batch to batch consistency of AB-101 need to 1) demonstrate antimicrobial or cicatrizant properties, 2) be present in AB-101, and 3) be detectable using analytical techniques. Using these criteria, the analytical efforts focused on 2 classes of compounds: polyphenols (proanthocyanidins) and alkaloids (taspine). Within the proanthocyanidin class, 4 specific compounds were focused on: catechin, epicatechin, gallocatechin, and epigallocatechin. The compound of importance within the alkaloid class is taspine. Each of these compounds fulfills the three required elements detailed above. The following are the chemical structures of the 5 compounds utilized as important markers for batch to batch consistency of AB-101.

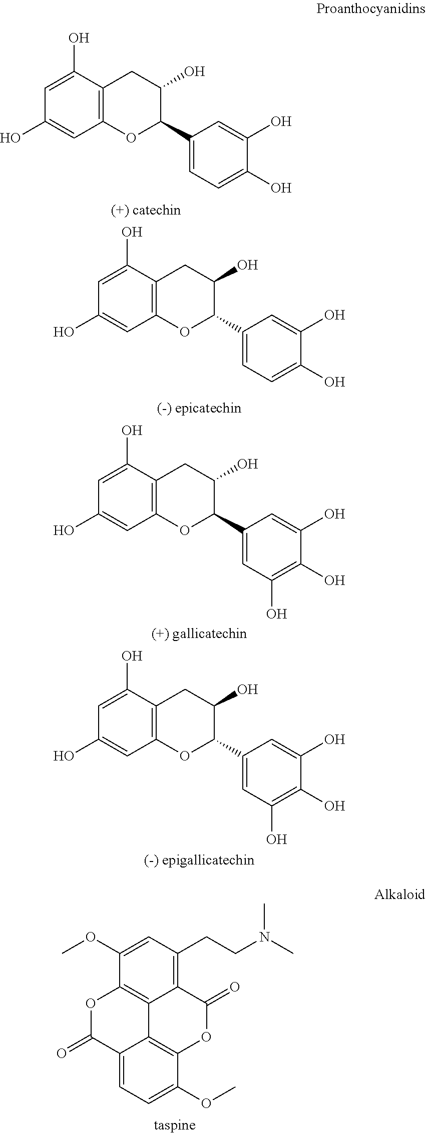 [SMILES: Oc1cc(O)c2c(c1)O[C@H](c1ccc(O)c(O)c1)[C@@H](O)C2 ; Oc1cc(O)c2c(c1)O[C@@H](c1ccc(O)c(O)c1)[C@H](O)C2 ; Oc1cc(O)c2c(c1)O[C@H](c1cc(O)c(O)c(O)c1)[C@@H](O)C2 ; Oc1cc(O)c2c(c1)O[C@@H](c1cc(O)c(O)c(O)c1)[C@H](O)C2 ; COc1ccc2c(=O)oc3c(OC)cc(CCN(C)C)c4c(=O)oc1c2c34]

For characterization studies, AB-101 extract was lyophilized and the lyophilized powder was subjected to three different extraction methods.

Method 1—Ultrasonic polyphenol extraction. The lyophilized AB-101 extract was dissolved into methanol. The resultant emulsion was then subjected to sonication for 10 minutes followed by centrifugation to remove particulates for 5 minutes. The supernatant was then subjected to LC-MS/MS analysis.

Method 2—Soxhlet extraction. The lyophilized AB-101 extract was subjected to a Soxhlet extraction with 80% ethanol. The ethanol was removed via a rotary evaporator. The resultant material was then subjected re-suspended in ethanol then subjected to LC-MS/MS analysis.

Method 3—Polyphenol extraction. The lyophilized AB-101 extract was incubated with methanol overnight at room temperature and in the dark. The supernatant was then filtered using Whatman filters, dried, and then re-suspended in methanol. The resultant material was then subjected to LC-MS/MS analysis.

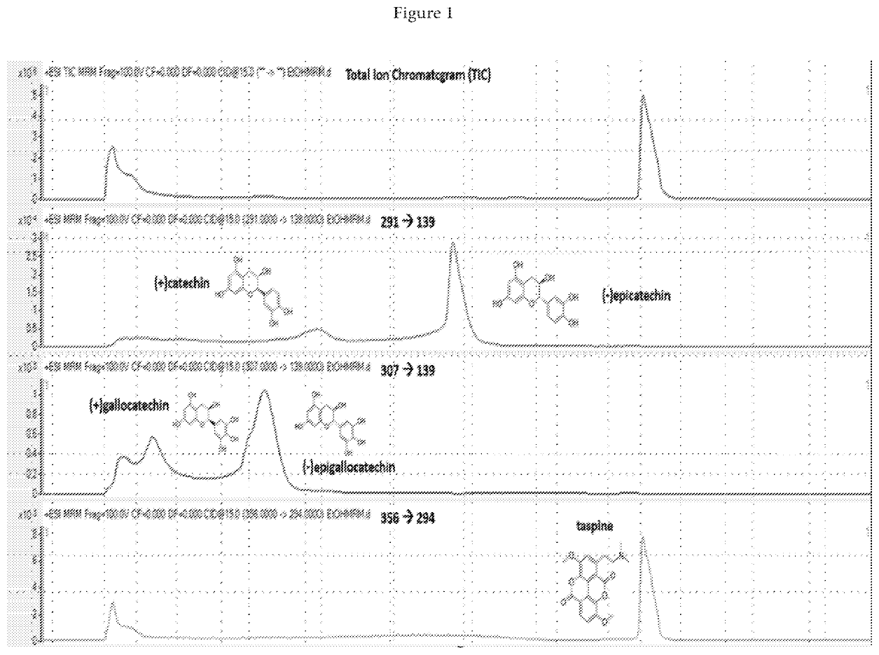

FIG. 1 depicts a representative Total Ion Chromatogram as well as additional Multiple Reaction Monitoring spectra that identify the important marker compounds in an AB-101 extract. The compounds are detectable using any of the three extraction methods.

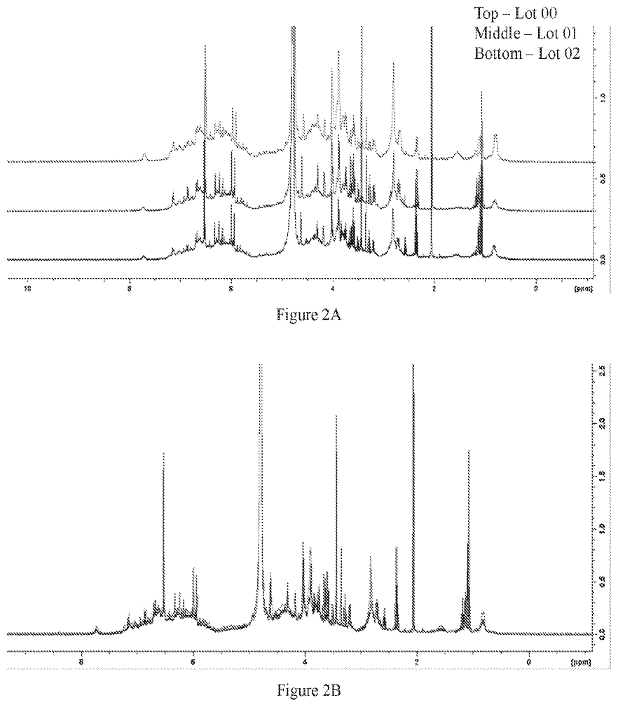

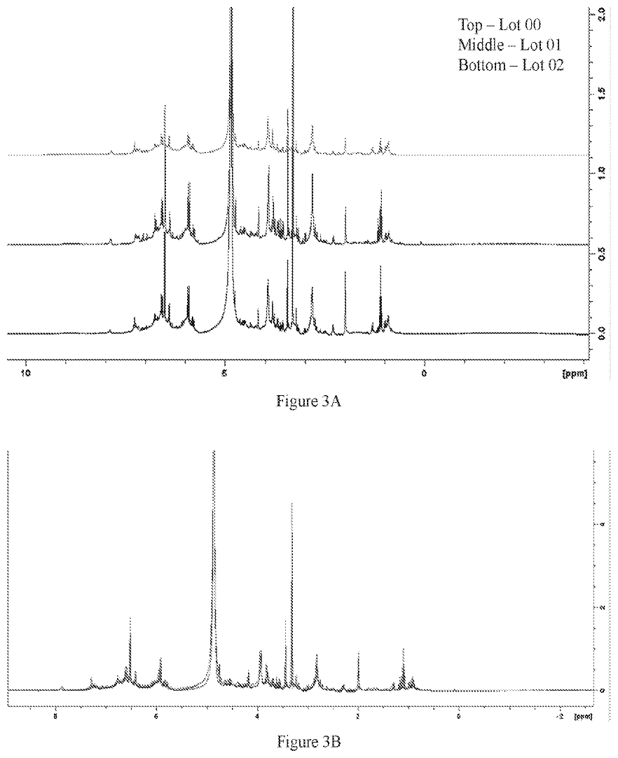

Biodiversity contributes to vast amounts of variability. In order to capture this variability, an NMR method utilizing a “spectral fingerprint” was used with an overlapping a reference standard. These fingerprinting captures most components within AB-101 and would be quantifiable using Nuclear Magnetic Resonance (NMR). Examples of NMR spectra using three different AB-101 lots (Lots 00, 01, and 02 respectively) and two different deuterated solvents (D₂O and d₄-Methanol respectively) are shown in FIGS. 2A and 3A with overlays of each solvents spectra being shown in FIGS. 2B and 3B and demonstrated no significant variability.

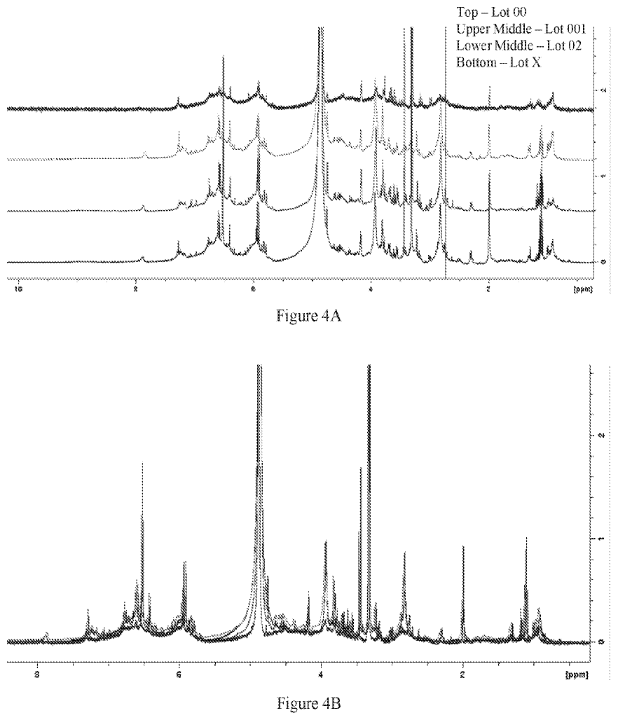

In another NMR analysis using the d₄-Methanol as the solvent, 4 distinct lots of AB-101 (Lots 00, 01, 02, and X respectively) are compared. NMR spectra of each lot are shown in FIG. 4A with overlays of each lots spectra being shown in FIG. 4B. While the fingerprint of the 4 lots looks similar, there are important differences. This is shown by comparing the concentration level in ppm based on LC-MS/MS Quantification and qualitative NMR “fingerprinting” on the marker compounds of catechin, epicatechin, gallocatechin, epigallocatechin, and taspine. The results are shown in Table 2 and indicate that lots 1 and 2 are more similar and lots X and 0 have the largest differences.

TABLE 2

| | AB-101 Lots Characterization | | | |
|---|---|---|---|---|
| | PPM (μg/g) | | | |
| Lot | X | 00 | 01 | 02 |
| Gallocatechin (GC) | 164.2 | 91.9 | 135.0 | 139.9 |
| Epigallocatechin (EGC) | 1357.6 | 380.7 | 1219.5 | 996.3 |
| Catechin (C) | 2.0 | 6.7 | 8.8 | 8.2 |
| Epicatechin (EC) | 2.6 | 5.2 | 8.3 | 6.1 |
| Taspine (T) | 50.4 | 43.4 | 50.1 | 51.1 |

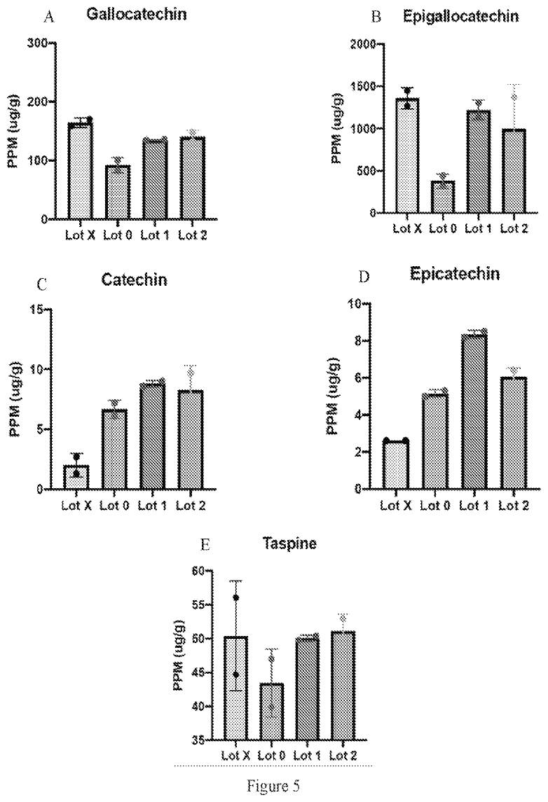

FIG. 5A-E depicts bar graphs comparing the AB-101 lot analysis results for each of the 5 marker compounds.

Lot 00 is an example of a lot that is not suitable for use in the pharmaceutical compositions and the methods of use described herein. Lots X, 01 and 02 are examples of lots that are suitable for use in the pharmaceutical compositions and the methods of use described herein.

In some embodiments the latex of Croton lechleri, preferably filtered latex of Croton lechleri, preferably filtered latex of Croton lechleri Müll.Arg. has a minimum bactericidal concentration (MBC) of about 6.25(% vol./vol.), about 12.5(% vol./vol.), about 25(% vol./vol.), about 50(% vol./vol.), or a range between any two of these values. In some embodiments the latex of Croton lechleri, preferably filtered latex of Croton lechleri, preferably filtered latex of Croton lechleri Müll.Arg. has a MBC of about 6.25(% vol./vol.) to about 50(% vol./vol.).

Some embodiments herein are directed to a pharmaceutical composition that further comprises one or more other therapeutic ingredients. In embodiments, the pharmaceutical composition comprises a therapeutically effective amount of the latex of Croton lechleri, preferably filtered latex of Croton lechleri, preferably filtered latex of Croton lechleri Müll.Arg. In embodiments, the pharmaceutical composition is suitable for topical administration or is a topical pharmaceutical composition.

The excipient(s) must be “acceptable” in the sense of being compatible with the other ingredients of the formulation and not deleterious to the recipient thereof. The excipient(s) will utilize a low number of known, well-characterized excipient ingredients that will not impart irritation or sensitization when used topically or in wounds or reduce the efficacy of AB-101. Proper formulation of the pharmaceutical composition is dependent upon the route of administration chosen. Any of the well-known techniques and excipients may be used as suitable and as understood in the art. The pharmaceutical compositions disclosed herein may be manufactured in any manner known in the art.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose, including eutectic solvents, eutectic-based ionic liquids, or ionic liquids. The pharmaceutical compositions can additionally include: lubricating agents such as talc, magnesium stearate, and mineral oil; wetting agents; emulsifying and suspending agents; preserving agents such as methyl- and propylhydroxy-benzoates.

The compositions include those suitable for topical (including, for example, dermal, nasal, oral mucosa, buccal, sublingual and intraocular) although the most suitable route may depend upon for example the condition and disorder of the recipient. The compositions may conveniently be presented in unit dosage form and may be prepared by any of the methods well known in the art of pharmacy. Typically, these methods include the step of bringing into association latex of Croton lechleri, preferably filtered latex of Croton lechleri, preferably filtered latex of Croton lechleri Müll.Arg. disclosed herein (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired composition.

The pharmaceutical compositions disclosed herein may be administered topically, that is by non-systemic administration. This includes the application of a composition disclosed herein externally to the surface of the skin and to achieve therapeutically effective amounts in the skin, such as the epidermis and/or dermis. In embodiments, topical administration or a topical pharmaceutical composition does not result in systemic administration or systemic exposure of the Croton lechleri to the patient.

In some embodiments, pharmaceutical compositions suitable for topical administration include liquid or semi-liquid preparations suitable for penetration through the skin to the site of inflammation such as a solution, powder, fluid emulsion, fluid suspension, semi-solid, ointment, paste, cream, gel, jelly, foam, liniment, lotion, and drops.

Lotions include those suitable for application to the skin. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid pharmaceutical compositions of the active ingredient for external application. They may be made by mixing the active ingredient in finely-divided or powdered form, alone or in solution or suspension in an aqueous or non-aqueous fluid, with the aid of suitable machinery, with a greasy or non-greasy base.

Preferred unit dosage pharmaceutical compositions are those containing an effective dose, as herein below recited, or an appropriate fraction thereof, of the active ingredient.

When employed as pharmaceuticals, the compositions can be administered in the form of pharmaceutical compositions. These compositions can be prepared in a manner well known in the pharmaceutical arts, and can be administered by a variety of routes, depending upon whether local or systemic treatment is desired and upon the area to be treated. Administration of the disclosed compositions may be topical (including dermal, nasal, oral mucosa, buccal, sublingual and intraocular). Pharmaceutical compositions for topical administration may include foams, transdermal patches, ointments, lotions, creams, gels, solutions, fluid emulsions, fluid suspensions, semi-solids, pastes, drops, suppositories, sprays, liquids, aerosolization, inhalers, and powders. Conventional pharmaceutical carriers, aqueous, powder or oily bases, thickeners and the like may be necessary or desirable. In some embodiments, the compositions can be contained in such pharmaceutical compositions with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The artisan can refer to various pharmacologic references for guidance.

In certain embodiments, the pharmaceutical composition is not a soap.

In certain embodiments, the pharmaceutical composition is liquid, ointment, lotion, or cream.

The pharmaceutical compositions can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active pharmaceutical compositions comprising latex of Croton lechleri, preferably filtered latex of Croton lechleri, preferably filtered latex of Croton lechleri Müll.Arg. can be effective over a wide dosage range and contain a therapeutically effective amount. It will be understood, however, that the amount of the pharmaceutical compositions comprising latex of Croton lechleri, preferably filtered latex of Croton lechleri, preferably filtered latex of Croton lechleri Müll.Arg. actually administered will usually be determined by a physician, according to the relevant circumstances, including the condition to be treated, the chosen route of administration, the actual composition administered, the age, weight, and response of the individual patient, the severity of the patient's symptoms, and the like.

The pharmaceutically acceptable excipient may be selected from one or more cream bases, one or more emulsifying agents, one or more preservatives, one or more humectants, one or more diluents, and latex of Croton lechleri, preferably filtered latex of Croton lechleri, preferably filtered latex of Croton lechleri Müll.Arg.

In some embodiments, the pharmaceutical composition may comprise about 0.01% to about 50% of latex of Croton lechleri, preferably filtered latex of Croton lechleri, preferably filtered latex of Croton lechleri Müll.Arg. disclosed herein. In some embodiments, the latex of Croton lechleri, preferably filtered latex of Croton lechleri, preferably filtered latex of Croton lechleri Müll.Arg. is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 300%, about 1% to about 295%, about 1% to about 290%, about 1% to about 285%, about 1% to about 280%, about 1% to about 275%, about 1% to about 270%, about 1% to about 265%, about 1% to about 260%, about 1% to about 255%, about 1% to about 250%, about 1% to about 245%, about 1% to about 240%, about 1% to about 235%, about 1% to about 230%, about 1% to about 225%, about 1% to about 220%, about 1% to about 215%, about 1% to about 210%, about 1% to about 205%, about 1% to about 200%, 195%, about 1% to about 190%, about 1% to about 185%, about 1% to about 180%, about 1% to about 175%, about 1% to about 170%, about 1% to about 165%, about 1% to about 160%, about 1% to about 155%, about 1% to about 150%, about 1% to about 145%, about 1% to about 140%, about 1% to about 135%, about 1% to about 130%, about 1% to about 125%, about 1% to about 120%, about 1% to about 115%, about 1% to about 110%, about 1% to about 105%, about 1% to about 100%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 2% to about 300%, about 2% to about 295%, about 2% to about 290%, about 2% to about 285%, about 2% to about 280%, about 2% to about 275%, about 2% to about 270%, about 2% to about 265%, about 2% to about 260%, about 2% to about 255%, about 2% to about 250%, about 2% to about 245%, about 2% to about 240%, about 2% to about 235%, about 2% to about 230%, about 2% to about 225%, about 2% to about 220%, about 2% to about 215%, about 2% to about 210%, about 2% to about 205%, about 2% to about 200%, 195%, about 2% to about 190%, about 2% to about 185%, about 2% to about 180%, about 2% to about 175%, about 2% to about 170%, about 2% to about 165%, about 2% to about 160%, about 2% to about 155%, about 2% to about 150%, about 2% to about 145%, about 2% to about 140%, about 2% to about 135%, about 2% to about 130%, about 2% to about 125%, about 2% to about 120%, about 2% to about 115%, about 2% to about 110%, about 2% to about 105%, about 2% to about 100%, about 2% to about 95%, about 2% to about 90%, about 2% to about 85%, about 2% to about 80%, about 2% to about 75%, about 2% to about 70%, about 2% to about 65%, about 2% to about 60%, about 2% to about 55%, about 2% to about 50%, about 2% to about 45%, about 2% to about 40%, about 2% to about 35%, about 2% to about 30%, about 2% to about 25%, about 2% to about 20%, about 2% to about 15%, about 3% to about 300%, about 3% to about 295%, about 3% to about 290%, about 3% to about 285%, about 3% to about 280%, about 3% to about 275%, about 3% to about 270%, about 3% to about 265%, about 3% to about 260%, about 3% to about 255%, about 3% to about 250%, about 3% to about 245%, about 3% to about 240%, about 3% to about 235%, about 3% to about 230%, about 3% to about 225%, about 3% to about 220%, about 3% to about 215%, about 3% to about 210%, about 3% to about 205%, about 3% to about 200%, 195%, about 3% to about 190%, about 3% to about 185%, about 3% to about 180%, about 3% to about 175%, about 3% to about 170%, about 3% to about 165%, about 3% to about 160%, about 3% to about 155%, about 3% to about 150%, about 3% to about 145%, about 3% to about 140%, about 3% to about 135%, about 3% to about 130%, about 3% to about 125%, about 3% to about 120%, about 3% to about 115%, about 3% to about 110%, about 3% to about 105%, about 3% to about 100%, about 3% to about 95%, about 3% to about 90%, about 3% to about 85%, about 3% to about 80%, about 3% to about 75%, about 3% to about 70%, about 3% to about 65%, about 3% to about 60%, about 3% to about 55%, about 3% to about 50%, about 3% to about 45%, about 3% to about 40%, about 3% to about 35%, about 3% to about 30%, about 3% to about 25%, about 3% to about 20%, about 3% to about 15%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, or a range between any two of these values. The forgoing percentages are relative to a composition made from AB-101 with exemplary amounts of the marker compounds present in the latex as disclosed in Table 1. To illustrate, a pharmaceutical compostion comprising 100% of AB-101 will contain at least about 110 PPM of gallocatechin, while a pharmaceutical compostion comprising 200% of AB-101 will contain at least about 220 PPM of gallocatechin. The foregoing all representing weight percentages of embodiments of the pharmaceutical compositions. In some embodiments, the pharmaceutical composition is suitable for topical administration (including, for example, dermal, nasal, oral mucosa, buccal, sublingual and intraocular).

In some embodiments, the latex of Croton lechleri, preferably filtered latex of Croton lechleri, preferably filtered latex of Croton lechleri Müll.Arg. is in a therapeutically effective amount. In some embodiments, the therapeutically effective amount may be in an amount of about 0.01% to about 100%, about 0.01% to about 95%, about 0.01% to about 90%, about 0.01% to about 85%, about 0.01% to about 80%, about 0.01% to about 75%, about 0.01% to about 70%, about 0.01% to about 65%, about 0.01% to about 60%, about 0.01% to about 55%, about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 100%, about 0.05% to about 95%, about 0.05% to about 90%, about 0.05% to about 85%, about 0.05% to about 80%, about 0.05% to about 75%, about 0.05% to about 70%, about 0.05% to about 65%, about 0.05% to about 60%, about 0.05% to about 55%, about 0.05% to about 50%, about 0.05% to about 45%, about 0.05% to about 40%, about 0.05% to about 30%, about 0.05% to about 20%, about 0.05% to about 10%, about 0.1% to about 100%, about 0.1% to about 95%, about 0.1% to about 90%, about 0.1% to about 85%, about 0.1% to about 80%, about 0.1% to about 75%, about 0.1% to about 70%, about 0.1% to about 65%, about 0.1% to about 60%, about 0.1% to about 55%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 300%, about 1% to about 295%, about 1% to about 290%, about 1% to about 285%, about 1% to about 280%, about 1% to about 275%, about 1% to about 270%, about 1% to about 265%, about 1% to about 260%, about 1% to about 255%, about 1% to about 250%, about 1% to about 245%, about 1% to about 240%, about 1% to about 235%, about 1% to about 230%, about 1% to about 225%, about 1% to about 220%, about 1% to about 215%, about 1% to about 210%, about 1% to about 205%, about 1% to about 200%, 195%, about 1% to about 190%, about 1% to about 185%, about 1% to about 180%, about 1% to about 175%, about 1% to about 170%, about 1% to about 165%, about 1% to about 160%, about 1% to about 155%, about 1% to about 150%, about 1% to about 145%, about 1% to about 140%, about 1% to about 135%, about 1% to about 130%, about 1% to about 125%, about 1% to about 120%, about 1% to about 115%, about 1% to about 110%, about 1% to about 105%, about 1% to about 100%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 2% to about 300%, about 2% to about 295%, about 2% to about 290%, about 2% to about 285%, about 2% to about 280%, about 2% to about 275%, about 2% to about 270%, about 2% to about 265%, about 2% to about 260%, about 2% to about 255%, about 2% to about 250%, about 2% to about 245%, about 2% to about 240%, about 2% to about 235%, about 2% to about 230%, about 2% to about 225%, about 2% to about 220%, about 2% to about 215%, about 2% to about 210%, about 2% to about 205%, about 2% to about 200%, 195%, about 2% to about 190%, about 2% to about 185%, about 2% to about 180%, about 2% to about 175%, about 2% to about 170%, about 2% to about 165%, about 2% to about 160%, about 2% to about 155%, about 2% to about 150%, about 2% to about 145%, about 2% to about 140%, about 2% to about 135%, about 2% to about 130%, about 2% to about 125%, about 2% to about 120%, about 2% to about 115%, about 2% to about 110%, about 2% to about 105%, about 2% to about 100%, about 2% to about 95%, about 2% to about 90%, about 2% to about 85%, about 2% to about 80%, about 2% to about 75%, about 2% to about 70%, about 2% to about 65%, about 2% to about 60%, about 2% to about 55%, about 2% to about 50%, about 2% to about 45%, about 2% to about 40%, about 2% to about 35%, about 2% to about 30%, about 2% to about 25%, about 2% to about 20%, about 2% to about 15%, about 3% to about 300%, about 3% to about 295%, about 3% to about 290%, about 3% to about 285%, about 3% to about 280%, about 3% to about 275%, about 3% to about 270%, about 3% to about 265%, about 3% to about 260%, about 3% to about 255%, about 3% to about 250%, about 3% to about 245%, about 3% to about 240%, about 3% to about 235%, about 3% to about 230%, about 3% to about 225%, about 3% to about 220%, about 3% to about 215%, about 3% to about 210%, about 3% to about 205%, about 3% to about 200%, 195%, about 3% to about 190%, about 3% to about 185%, about 3% to about 180%, about 3% to about 175%, about 3% to about 170%, about 3% to about 165%, about 3% to about 160%, about 3% to about 155%, about 3% to about 150%, about 3% to about 145%, about 3% to about 140%, about 3% to about 135%, about 3% to about 130%, about 3% to about 125%, about 3% to about 120%, about 3% to about 115%, about 3% to about 110%, about 3% to about 105%, about 3% to about 100%, about 3% to about 95%, about 3% to about 90%, about 3% to about 85%, about 3% to about 80%, about 3% to about 75%, about 3% to about 70%, about 3% to about 65%, about 3% to about 60%, about 3% to about 55%, about 3% to about 50%, about 3% to about 45%, about 3% to about 40%, about 3% to about 35%, about 3% to about 30%, about 3% to about 25%, about 3% to about 20%, about 3% to about 15%, about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 3%, about 5%, about 10%, about 15%, about 20%, about 25%, about 30%, about 35%, about 40%, about 45%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, or a range between any two of these values. The forgoing percentages are relative to a compostion made from AB-101 with exemplary amounts of the marker compounds present in the latex as disclosed in Table 1. To illustrate, a therapeutically effective amount in the amount of 100% of AB-101 will contain at least about 110 PPM of gallocatechin, while a therapeutically effective amount in the amount of 200% of AB-101 will contain at least about 220 PPM of gallocatechin. The foregoing all representing weight percentages of the pharmaceutical composition.

In some embodiments, the therapeutically effective amount can vary according to, for example, the particular use for which the treatment is made, the manner of administration of the composition, the health and condition of the patient, and the judgment of the prescribing physician. The proportion or concentration of latex of Croton lechleri, preferably filtered latex of Croton lechleri, preferably filtered latex of Croton lechleri Müll.Arg. in a pharmaceutical composition can vary depending upon a number of factors including dosage, chemical characteristics (e.g., hydrophobicity), and the route of administration. For example, the compositions can be provided in an aqueous physiological buffer solution containing about 0.1 to about 10% w/v of the composition for parenteral administration. Some typical dose ranges for the compositions are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the composition selected, composition of the excipient, and its route of administration. Effective doses can be extrapolated from dose-response curves derived from in vitro or animal model test systems.

The amount of composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications.

In certain embodiments the one or more cream bases is selected from cetyl alcohol, isopropylmeristat, petroleum jelly, or any combination thereof. In some embodiments, the one or more cream bases is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, or a range between any two of these values. The foregoing all representing weight percentages of the pharmaceutical composition. In some embodiments, the pharmaceutical composition is suitable for topical administration (including, for example, dermal, nasal, oral mucosa, buccal, sublingual and intraocular).

In certain embodiments the one or more emulsifying agents is selected from span20, tween80, or any combination thereof. In some embodiments, the one or more emulsifying agents is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, or a range between any two of these values. The foregoing all representing weight percentages of the pharmaceutical composition. In some embodiments, the pharmaceutical composition is suitable for topical administration (including, for example, dermal, nasal, oral mucosa, buccal, sublingual and intraocular).

In certain embodiments the one or more preservatives is selected from propylparaben, methylparaben, or any combination thereof. In some embodiments, the one or more preservatives is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, or a range between any two of these values. The foregoing all representing weight percentages of the pharmaceutical composition. In some embodiments, the pharmaceutical composition is suitable for topical administration (including, for example, dermal, nasal, oral mucosa, buccal, sublingual and intraocular).

In certain embodiments the one or more humectants is propylene glycol. In some embodiments, the one or more humectants is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, or a range between any two of these values. The foregoing all representing weight percentages of the pharmaceutical composition. In some embodiments, the pharmaceutical composition is suitable for topical administration (including, for example, dermal, nasal, oral mucosa, buccal, sublingual and intraocular).

In certain embodiments the one or more diluents is water. Wherein the one or more diluents is in a quantity sufficient to bring the sum of the component weight percentages of the pharmaceutical composition to 100%.

The one or more ethanolic extracts of Croton lechleri is prepared by dissolving the latex of Croton lechleri, preferably filtered latex of Croton lechleri, preferably filtered latex of Croton lechleri Müll.Arg. in ethanol. The latex of Croton lechleri, preferably filtered latex of Croton lechleri, preferably filtered latex of Croton lechleri Müll.Arg. tree is not modified prior to dissolving in ethanol.

In some embodiments the pharmaceutical composition comprises about 10.0% cetyl alcohol, about 7.0% isopropylmeristat, about 21.0% petroleum jelly, about 1.5% span20, about 1.5% tween 80, about 0.02% propylparaben, about 0.18% methylparaben, about 5% propylene glycol, about 15% one or more ethanolic extracts of Croton lechleri, and water in a quantity sufficient to bring the sum of the component weight percentages of the pharmaceutical composition to 100%.

### Methods of Use

Skin infections occur when the bacteria penetrate the skin and spread resulting in one or more of pain, swelling, other types of discomfort, and skin color changes and include but are not limited to uncomplicated or complicated skin infections, mild or serious skin infection, Acute Bacterial Skin and Skin Structure Infections (or ABSSSIs which are any lesion 75 cm² or larger), lesser skin infections called Secondarily Infected Traumatic Lesions (or SITLs), Skin and Soft Tissue Infections (or SSTIs), primary infections as exampled by all forms impetigo including but not limited to Mupirocin-resistant impetigo, or skin infections caused by drug resistant bacteria. Some bacteria have become resistant to commonly used antibiotics or drugs. Antibiotic or drug resistant bacteria are bacteria that are not controlled or killed by antibiotics or drugs. They are able to survive and even multiply in the presence of an antibiotic or drug. Most infection-causing bacteria can become resistant to at least some antibiotics or drugs. Bacteria that are resistant to many antibiotics are known as multi-resistant organisms (or MRO) or multi-drug resistant organisms (or MDRO). Treating skin infections topically can be a first line of defense to prevent the skin infection from spreading to systemic or internal body infection preventing detrimental and serious health effects including death.

The present invention relate to methods of treatment of acute bacterial skin or skin structure infections in a subject comprising the administration of a therapeutically effective amount of latex of Croton lechleri, preferably filtered latex of Croton lechleri, preferably filtered latex of Croton lechleri Müll.Arg. or a pharmaceutical composition containing latex of Croton lechleri, preferably filtered latex of Croton lechleri, preferably filtered latex of Croton lechleri Müll.Arg. as disclosed herein. In embodiments, the pharmaceutical composition may include a pharmaceutically acceptable excipient. As disclosed herein the latex of Croton lechleri, preferably filtered latex of Croton lechleri, preferably filtered latex of Croton lechleri Müll.Arg. shall comprise one or more compounds selected from: gallocatechin, epigallocatechin, catechin, epicatechin, and taspine, and combinations thereof. Each of gallocatechin, epigallocatechin, catechin, epicatechin, and taspine may be present in the latex of Croton lechleri, preferably filtered latex of Croton lechleri, preferably filtered latex of Croton lechleri Müll.Arg. in the amounts found in Table 1 or paragraphs [0047]-[0051], or any combination of such amounts.

Also provided herein is latex of Croton lechleri, preferably filtered latex of Croton lechleri, preferably filtered latex of Croton lechleri Müll.Arg. as disclosed herein for use as a medicament.

Also provided herein is latex of Croton lechleri, preferably filtered latex of Croton lechleri, preferably filtered latex of Croton lechleri Müll.Arg. as disclosed herein for use as a medicament for the treatment of acute bacterial skin or skin structure infections.

Also provided is the use of latex of Croton lechleri, preferably filtered latex of Croton lechleri, preferably filtered latex of Croton lechleri Müll.Arg. as disclosed herein as a medicament for the treatment of acute bacterial skin or skin structure infections.

Also provided is latex of Croton lechleri, preferably filtered latex of Croton lechleri, preferably filtered latex of Croton lechleri Müll.Arg. as disclosed herein for use in the manufacture of a medicament for the treatment of acute bacterial skin or skin structure infections.

Also provided is the use of latex of Croton lechleri, preferably filtered latex of Croton lechleri, preferably filtered latex of Croton lechleri Müll.Arg. as disclosed herein for the treatment of acute bacterial skin or skin structure infections.

Also provided herein is a method of treating acute bacterial skin or skin structure infections comprising contacting acute bacterial skin or skin structure infections with latex of Croton lechleri, preferably filtered latex of Croton lechleri, preferably filtered latex of Croton lechleri Müll.Arg. as disclosed herein.

Also provided herein is a method for achieving a therapeutic effect in a patient comprising the administration of a therapeutically effective amount of latex of Croton lechleri, preferably filtered latex of Croton lechleri, preferably filtered latex of Croton lechleri Müll.Arg.

In certain embodiments, the acute bacterial skin or skin structure infection is selected from the group consisting of Streptococcus pyogenes infection, Staphylococcus aureus infection, methicillin-resistant Staphylococcus aureus (MRSA) infection, Mupirocin-resistant MRSA, Enterococcus faecalis infection, Gram-positive bacteria infection, Gram-negative bacteria infection, cellulitis/erysipelas, wound infection, burn infection, major cutaneous abscesses, impetigo, Mupirocin-resistant impetigo, Vancomycin resistant bacteria infection, Mupirocin resistant bacteria infection, Clostridium difficile infection, drug-resistant Neisseria gonorrhoeae infection, Streptococcus pneumoniae infection, drug-resistant Streptococcus pneumoniae infection, drug-resistant Klebsiella pneumoniae infection, drug-resistant Malaria infection, Multi-drug resistant (MDR) infection, Extensively drug-resistant (XDR) Tuberculosis infection, Escherichia coli (E. coli) infection, Shiga toxin-producing Escherichia coli (E. coli) infection, infections caused by bacteria possessing Enzyme NDM-1 (New Delhi Metallo-beta-lactamase-1), Clostridium difficile infection, Enterococcus infection, Mycobacterium tuberculosis infection, Mycoplasma genitalium infection, Streptococcus infection, Campylobacter infection, Neisseria gonorrhoeae infection, Gamma proteobacteria infection, Enterobacteriaceae infection, Carbapenem-Resistant Enterobacteriaceae, infection, Klebsiella pneumoniae infection, Salmonella infection, E. coli infection, Pseudomonadales infection, Acinetobacter infection, Pseudomonas aeruginosa infection, MDR Pseudomonas aeruginosa infection, Coagulase-negative Staphylococcus infection, and combinations thereof.

In certain embodiments, the acute bacterial skin or skin structure infection is selected from the group consisting of Streptococcus pyogenes infection, Staphylococcus aureus infection, methicillin-resistant Staphylococcus aureus (MRSA) infection, Mupirocin-resistant MRSA, Enterococcus faecalis infection, Streptococcus pneumoniae infection, Escherichia coli (E. coli) infection, Pseudomonas aeruginosa infection, MDR Pseudomonas aeruginosa infection, Coagulase-negative Staphylococcus infection, and combinations thereof.

In certain embodiments, the acute bacterial skin or skin structure infection is methicillin-resistant Staphylococcus aureus infection.

In certain embodiments, the acute bacterial skin or skin structure infection is Mupirocin-resistant MRSA infection.

In certain embodiments, the acute bacterial skin or skin structure infection is Pseudomonas aeruginosa infection.

In certain embodiments, the acute bacterial skin or skin structure infection is MDR resistant Pseudomonas aeruginosa infection.

In certain embodiments, the acute bacterial skin or skin structure infection is not methicillin-resistant Staphylococcus aureus (MRSA) infection.

The pharmaceutical compositions may be administered in various modes, e.g. topical (including, for example, dermal, nasal, oral mucosa, buccal, sublingual and intraocular). Also, the route of administration may vary depending on the condition and its severity.

Pharmaceutical compositions of the present invention may be administered once per day, twice per day, thrice per day, 4 times per day, 5 times per day, 6 times per day, 7 times per day, 8 times per day, 9 times per day, 10 times per day, or a range between of these values. In some embodiments, the pharmaceutical composition is administered twice per day. In some embodiments, the pharmaceutical composition is administered thrice per day. In some embodiments, the pharmaceutical composition is administered until the acute bacterial skin or skin structure infection is resolved, gone, or treated.

Pharmaceutical compositions of the present invention may be administered continuously, every 15 minutes 30 min , 1 hour(s) (hr.), 1½ hr., 2 hr., 2½ hr., 3 hr., 4 hr., 6 hr., 8 hr., 12 hr., 24 hr., 36 hr., 48 hr., 3 days, 4 days, 5 days, 6, days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or a range between of these values. In some embodiments, the administration lasts 24 weeks. In particular embodiments, the administration lasts 2 weeks. In some embodiments, the administration lasts until the acute bacterial skin or skin structure infection is resolved, gone, or treated.

Treatment of the acute bacterial skin or skin structure infections will last 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or a range between of these values. In some embodiments, the treatment lasts 2 weeks. In some embodiments, the treatment lasts until the acute bacterial skin or skin structure infection is resolved, gone, or treated.

Treatment of the acute bacterial skin or skin structure infections may continue until complete resolution of the target lesion.

Treatment of the acute bacterial skin or skin structure infections may continue at the discretion of the prescribing physician.

In certain embodiments, the pharmaceutical compositions of the present invention may be topically applied directly to the acute bacterial skin or skin structure infection.

In certain embodiments, the pharmaceutical compositions of the present invention may be topically applied directly to the lesion that results from an acute bacterial skin or skin structure infection.

In certain embodiments, dosage is 1-2 drops of a pharmaceutical compositions of the present invention per acute bacterial skin or skin structure infection, once, twice or more daily with the composition applied to each acute bacterial skin or skin structure infection. Multiple drops are applied to a crop of lesions. The drops are allowed to dry (several minutes) or they are gently rubbed (about 15 seconds) over the acute bacterial skin or skin structure infection until the composition changes to a “creamier” state. It then dries very quickly (several seconds).

In certain embodiments, the pharmaceutical compositions of the present invention is first applied to a bandage (e.g., gauze), which is then applied to the acute bacterial skin or skin structure infection. The treated bandage is applied to each lesion. If the bandage is separated from the lesion or if the dressing has been worn for 24 hours, a new, treated bandage may be applied. A new dressing is generally, but not always, applied every day and may be applied up to once per week or longer period of time. In one embodiment, the composition is administered until the symptoms (e.g., skin lesions) disappear, become less pronounced, or problematic side effects occur.

In some embodiments, the pharmaceutical compostion has a minimum inhibitory concentration (MIC) of at least about 0.01%, at least about 0.05%, at least about 0.1%, at least about 0.25%, at least about 0.5%, at least about 0.75%, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%.

### EXAMPLE 1

### Evaluation of AB-101 Antibacterial Activity

An Agar Well Diffusion Assay pilot evaluation of AB-101 BRM was conducted to seek an initial signal of the activity of AB-101 BRM against various bacteria. AB-101 BRM was tested against the following bacterial species:
Streptococcus pneumoniae (ATCC 49619) (SPNEU) Gram (+)
Streptococcus pyogenes (clinical isolate) (GABHS) Gram (+)
Enterococcus faecalis (ATCC 29212) (EFAECL) Gram (+)
Coagulase-negative Staphylococcus (clinical isolate) (CNS) Gram (+)
Escherichia coli (ATCC 25922) (EC) Gram (−)
Pseudomonas aeruginosa (ATCC 27853) (PAER) Gram (−)
Klebsiella pneumoniae (ATCC 700603) (KPNEU) Gram (−)
Staphylococcus aureus (ATCC 52923) (SAUR) Gram (+)
Methicillin-resistant Staphylococcus aureus (ATCC 49476) (MRSA) Gram (+)

Isolates were subcultured the day before the Agar Well Diffusion Assay was performed. On the day of the assay, a bacterial suspension (0.08-0.12 MacFarland units) of each bacteria isolate was made in Trypticase Soy Broth (TSB), and these suspensions were used to plate a lawn of bacterial organisms on Mueller-Hinton agar or Mueller-Hinton sheep's blood agar. A control antibiotic Kirby-Bauer (K-B) disk plus K-B disks containing 25 uL of AB-101 dilutions were placed on the plates, and the plates were incubated overnight. The next day, the width of the zone of growth inhibition surrounding each K-B disk was recorded (in mm). The larger the number, the greater the zone of inhibition, and the greater the inhibition of growth of the target organisms.

AB-101 was used undiluted (AB⁰) and in 10-fold dilutions (AB⁻¹, AB⁻², AB⁻³, AB⁻⁴) made in dimethylsulfoxide (DMSO). Control experiments showed that AB-101 BRM precipitated when mixed into either TSB or Hanks Balanced Salt Solution with Ca⁺⁺ and Mg⁺⁺, but was apparently soluble in DMSO, ethanol or methanol. However, when initially diluted into DMSO, EtOH or MeOH at 1:10 and subsequently diluted into TSB, AB-101 continued to precipitate. Therefore, all dilutions were made into DMSO and a K-B disk containing 25 uL DMSO was included in all experiments as a vehicle control. Table 3 provides a summary of the results. Table 4 (AMP=Ampicillin; GM=Gentamicin) provides the individual results of the Zone of Inhibition Assay for AB-101. As shown in Table 3 AB-101 is effective against both Gram (+) and Gram (−) bacteria.

TABLE 3

| Bacteria | Abbreviation | Bacteria Type | AB-101 Activity |
|---|---|---|---|
| Streptococcus pneumoniae | (SPNEU) | Gram (+) | Yes |
| Streptococcus pyogenes | (GABHS) | Gram (+) | Yes |
| Enterococcus faecalis | (EFAECL) | Gram (+) | Yes |
| Coagulase-negative Staphylococcus | (CNS) | Gram (+) | Yes |
| Escherichia coli | (EC) | Gram (−) | Yes |
| Pseudomonas aeruginosa | (PAER) | Gram (−) | Yes |
| Klebsiella pneumoniae | (KPNEU) | Gram (−) | Unknown |
| Staphylococcus aureus | (SAUR) | Gram (+) | Yes |
| Methicillin-resistant | (MRSA) | Gram (+) | Yes |
| Staphylococcus aureus | | | |

TABLE 4

| | Antibiotic | Zone of Inhibition (MM) | | | | | |
|---|---|---|---|---|---|---|---|
| | Controls | DMSO | AB⁰ | AB⁻¹ | AB⁻² | AB⁻³ | AB⁻⁴ |
| SPNEU | Gram (+) | | | | | | |
| Jun. 3, 2005 | AMP 14 | 0 | 2 | 1 | <1 | <1 | <1 |
| Jun. 6, 2005 | AMP 13 | 1 | 4 | 1 | <1 | <1 | <1 |
| Jun. 7, 2005 | AMP no growth | | | | | | |
| GABHS | Gram (+) | | | | | | |
| Jun. 3, 2005 | AMP 13 | 0 | 2 | 1 | <1 | <1 | <1 |
| Jun. 6, 2005 | AMP 16 | <1 | 1 | <1 | Error | Error | Error |
| Jun. 7, 2005 | AMP 13 | <1 | 2 | 1 | 1 | <1 | <1 |
| EFAECL | Gram (+) | | | | | | |
| Jun. 3, 2005 | AMP 8 | 0 | 3 | 2 | 1 | <1 | <1 |
| Jun. 6, 2005 | GM 5 | <1 | 2 | 1 | <1 | <1 | <1 |
| Jun. 7, 2005 | AMP 8 | <1 | 3 | 1 | <1 | <1 | <1 |
| CNS | Gram (+) | | | | | | |
| Jun. 6, 2005 | GM 11 | <1 | 4 | 4 | 1 | <1 | <1 |
| Jun. 7, 2005 | GM 13 | <1 | 5 | 4 | 1 | <1 | <1 |
| EC | Gram (−) | | | | | | |
| Jun. 3, 2005 | GM 9 | 0 | <1 | 4 | 2 | <1 | <1 |
| Jun. 6, 2005 | GM 8 | <1 | ppt | 5 | 1 | <1 | <1 |
| Jun. 7, 2005 | GM 7 | <1 | <1 | 1 | <1 | <1 | <1 |
| PAER | Gram (−) | | | | | | |
| Jun. 3, 2005 | GM 7 | 0 | 4 | 1 | 1 | <1 | <1 |
| Jun. 6, 2005 | GM 10 | <1 | 1 | 1 | <1 | <1 | <1 |
| Jun. 7, 2005 | GM 8 | <1 | <1 | <1 | <1 | <1 | <1 |
| KPNEU | Gram (−) | | | | | | |
| Jun. 3, 2005 | GM 4 | 0 | <1 | <1 | <1 | <1 | <1 |
| Jun. 6, 2005 | GM 4 | <1 | <1 | <1 | <1 | <1 | <1 |
| Jun. 7, 2005 | GM 5 | <1 | <1 | <1 | <1 | <1 | <1 |
| SAUR | Gram (+) | | | | | | |
| Jun. 2, 2005 | GM 6 | 0 | 3 | 2 | <1 | <1 | <1 |
| Jun. 3, 2005 | GM 7 | 0 | 3 | 2 | <1 | <1 | <1 |
| Jun. 6, 2005 | GM 8 | <1 | 3 | 3 | 1 | <1 | <1 |
| Jun. 7, 2005 | GM 8 | 0 | 4 | 4 | <1 | <1 | <1 |
| MRSA | Gram (+) | | | | | | |
| Jun. 3, 2005 | GM 1 | 0 | 5 | 3 | <1 | <1 | <1 |
| Jun. 6, 2005 | GM <1 | <1 | 4 | 4 | <1 | <1 | <1 |
| Jun. 7, 2005 | GM 1 | 0 | 5 | 4 | <1 | <1 | <1 |

When evaluating the preliminary K-B data, there was a dilution effect on inhibition which validated this testing. The zones of inhibition are 4-5 mm at the highest concentration, which is indicative of antibacterial activity especially since no standard for what constitutes activity exists for the compounds in AB-101. When assessing antibiotic effects in a K-B type test, the minimum zone of inhibition for considering a positive test varies depending on the antibiotic tested with anything more than 0 mm considered positive in some cases or as much as 10 mm or more needed for others. The laboratory concluded that with AB-101, any inhibition should be considered a preliminary positive test and confirmed with more rigorous testing, and that AB-101's actual antibacterial activity may be greater than what was seen in these experiments. AB-101 was insoluble in aqueous buffer which likely limited its diffusion into agar further indicating anything more than 0 mm for AB-101 should be considered positive.

As shown in Table 4, AB-101 had the greatest activity against the following Gram-positive organisms: Staphylococci>Streptococci & Enterococcus faecalis. Data for AB-101 activity against Gram-negative rods suggests activity against Pseudomonas aeruginosa and E. coli, but minimal activity against Klebsiella.

### EXAMPLE 2

### Antibacterial Testing of AB-101 against Staphylococcus aureus

AB-101 was tested against an extensive variety of bacterial strains, including Mupirocin-resistant MRSA, Mupirocin-resistant MSSA, Mupirocin-susceptible MSSA, Mupirocin-susceptible MRSA, and other skin-related pathogens.

The initial antimicrobial testing of AB-101 is described below. The efficacy of AB-101 was assessed against ATCC 29213, an MSSA strain and ATCC 33591, an MRSA strain, using a variety of different microbiological assays in an attempt to identify the best approach at determining AB-101 sensitivity.

The first assay performed was the agar diffusion assay. A lawn of bacteria was painted on an agar plate, and the surface was then gently incised with a 6 mm biopsy punch. A 10 μL droplet of sample was then applied inside the ring incised by the biopsy punch. Three lots of AB-101 were assessed and test conditions were conducted in duplicates. After the agar plates were incubated, the diameter of the Zone of Inhibition (ZOI) was measured with a ruler and the average diameter was calculated; for samples where the ZOI was not circular, the diameter of the narrowest portion of the ZOI was measured. Table 5 provides the average zone of inhibition of AB-101 resin in the agar diffusion assay.

TABLE 5

| | Average zone of inhibition (mm) of AB-101 resin | | |
|---|---|---|---|
| Strain | AB-101 Lot 00 | AB-101 Lot 01 | AB-101 Lot 02 |
| ATCC 29213 (MSSA) | 8.0 | 8.5 | 7.5 |
| ATCC 33591 (MRSA) | 8.5 | 9.0 | 9.0 |

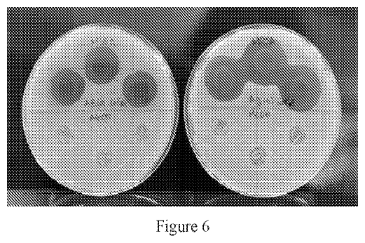

The ZOI for AB-101 was around 7.5 to 9.0 mm and were similar between MRSA and MSSA. The surface of AB-101 droplets tend to form a film after exposure to air, resulting in higher surface tension and limiting its ability to flow and spread across the surface of the agar. Extractions of raw, botanical material are often performed prior to antimicrobial testing. A methanol extraction of AB-101 by lyophilizing the resin to remove its water content, then resuspending the dried powder to 250 mg/mL with pure methanol. Each lot of methanol extracted AB-101 was then tested in an agar diffusion assay in triplicates, and methanol-only controls were included for each strain to assess the antimicrobial contribution of the solvent. The average ZOI of the methanol extracted AB-101 is summarized in Table 6. A representative image of the ZOI of methanol extracted AB-101 against MSSA (on the left) and MRSA (on the right) is shown in FIG. 6, with the methanol extract on the top of the plates and the methanol controls on the bottom of the plates.

TABLE 6

| | Average zone of inhibition (mm) of methanol extracted AB-101 | | | |
|---|---|---|---|---|
| | | | | Methanol |
| Strain | AB-101 Lot 00 | AB-101 Lot 01 | AB-101 Lot 02 | control |
| ATCC 29213 (MSSA) | 17.3 | 18.3 | 16.7 | 9.1 |
| ATCC 33591 (MRSA) | 20.0 | 23.7 | 20.7 | 8.8 |

The average ZOI of methanol extracted AB-101 were between 16.7 to 23.7 mm, roughly double that of the methanol-only control (Table 7). In addition, the ZOI of the methanol control as seen in Table 7 is hazy, indicating the methanol evaporated quickly after being dispensed on the agar and did not greatly inhibit bacterial growth. This contrasts with methanol extracted AB-101, which exhibit very clear ZOI, indicative of a strong antimicrobial effect from AB-101.

In addition to agar diffusion assay, the antimicrobial activity of AB-101 was assessed in broth microdilution assay. Both AB-101 and methanol extracted AB-101 were tested by 2-fold serial dilutions into the bacteriological media, Cation-Adjusted Mueller-Hinton Broth (CAMHB). An equal volume of bacteria inoculum was then added to the serially diluted samples in a 96 well plate to generate the final test concentrations in Table 7. A methanol-only control was also included to determine the contribution of the solvent for the methanol extracted AB-101. All conditions were tested in duplicates and the 96 well plates were incubated overnight for approximately 20 hours at 37 ° C.

TABLE 7
Concentration of AB-101 and methanol extracted AB-101 tested in broth
microdilution assay

| Well # | 1 | 2 | 3 | 4 | 5 | 6 | 7 | 8 | 9 | 10 | 11 | 12 |
|---|---|---|---|---|---|---|---|---|---|---|---|---|
| AB-101 (%) | 50 | 25 | 12.5 | 6.25 | 3.125 | 1.563 | 0.781 | 0.391 | 0.195 | 0.098 | 0.049 | 0 |
| Methanol AB- | 125 | 62.5 | 31.25 | 15.625 | 7.8125 | 3.906 | 1.953 | 0.977 | 0.488 | 0.244 | 0.122 | 0 |
| 101 (mg/mL) | | | | | | | | | | | | |
| Methanol | 50 | 25 | 12.5 | 6.25 | 3.125 | 1.563 | 0.781 | 0.391 | 0.195 | 0.098 | 0.049 | 0 |
| control (%) | | | | | | | | | | | | |

Both AB-101 and the methanol extracted AB-101 formed cloudy, brownish precipitates in the presence of the water-based CAMHB media. To assess the viability of bacteria in the cloudy suspension, 10 μL from each assay condition was drop-platted on agar and incubated to determine the Minimum Bactericidal Concentration (MBC), which is defined as the lowest concentration of a test solution from which no colonies are recovered after incubation. The MBC values were determined below in Table 8. The MBC value of the methanol extracted AB-101 is considerable lower than the methanol-only control, a result that is similar to the agar diffusion assay performed with this form of AB-101 (see Table 8), and reinforces the observation that methanol extracted AB-101 has potent antimicrobial efficacy against Staphylococcus aureus, which includes MRSA, Mupirocin-resistant MRSA and MSSA that is independent from the solvent.

TABLE 8
MBC values of AB-101 and methanol extracted AB-101 tested in broth
microdilution assay

| | AB-101 | | Methanol Extracted AB-101 | | Methanol control MBC | |
|---|---|---|---|---|---|---|
| Strain | MBC (%) | | MBC (mg/mL) | | (%) | |
| MSSA | 50 | 50 | 0.977 | 0.977 | 50 | 50 |
| (ATCC 29213) | | | | | | |
| MRSA | 50 | 50 | 0.977 | 0.977 | 50 | 50 |
| (ATCC 33591) | | | | | | |

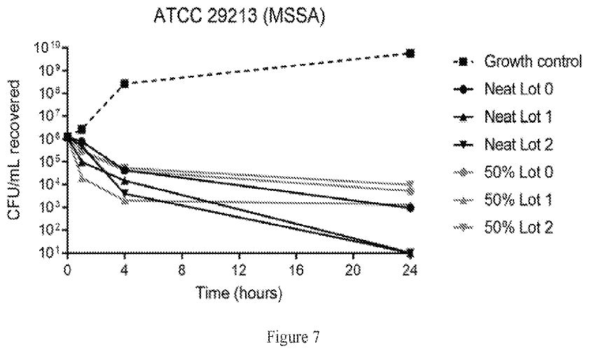

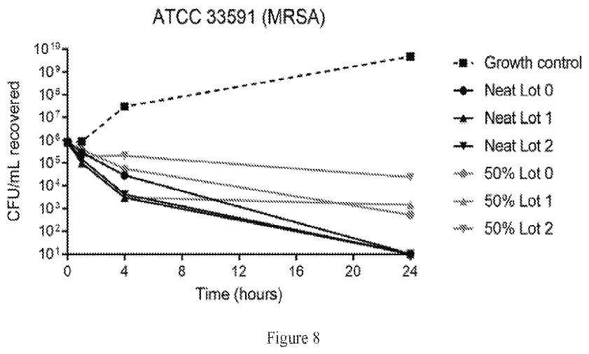

While the precipitation of AB-101, both in BRM and methanol extracted form, possesses a significant technical challenge for rapid antimicrobial screening, we noticed that the MBC value for AB-101 BRM is 50%. This indicates AB-101 is bactericidal in the range of 100% to 50% within 20 hours of contact with S. aureus. To determine how quickly and to what extent AB-101 exerts its bactericidal effect, a time-kill kinetics assay was performed against MRSA and MSSA. Undiluted AB-101 BRM or AB-101 diluted to 50% (v/v) with CAMHB were inoculated with MSSA or MRSA. Bacteria density was enumerated on agar plates at 1, 4, and 24 hours after inoculation and graphed relative to the time 0 inoculum density. The CFU/mL recovered and graphed data for MSSA (Table 9 and FIG. 7) and MRSE (Table 10 and FIG. 8) are shown below. In Table 9 CFU/mL values for Undiluted AB-101 BRM Lot 001 at T24 and Undiluted AB-101 BRM Lot 002 at T24 and in Table 10 CFU/mL values for Undiluted AB-101 BRM Lot 00 at T24, Undiluted AB-101 BRM Lot 001 at T24 and Undiluted AB-101 BRM Lot 002 at T24 indicate samples from which no colonies were recovered and represents the limit of detection of the assay (ie, 10 CFU/mL).

TABLE 9
MSSA CFU/mL recovered in time-kill assay

| | CFU/mL recovered | | | |
|---|---|---|---|---|
| ATCC 29213 (MSSA) | T0 | T1 | T4 | T24 |
| Growth control | 1.2E+06 | 2.7E+06 | 2.7E+08 | 5.8E+09 |
| Undiluted AB-101 Lot 00 | | 8.0E+05 | 4.3E+04 | 9.3E+02 |
| Undiluted AB-101 Lot 01 | | 1.0E+05 | 1.5E+04 | 1.0E+01 |
| Undiluted AB-101 Lot 02 | | 5.0E+05 | 4.0E+03 | 1.0E+01 |
| Diluted AB-101 50% Lot 00 | | 3.0E+05 | 4.1E+04 | 5.2E+03 |
| Diluted AB-101 50% Lot 01 | | 2.0E+04 | 2.0E+03 | 1.3E+03 |
| Diluted AB-101 50% Lot 02 | | 3.0E+05 | 5.4E+04 | 9.7E+03 |

TABLE 10
MRSA CFU/mL recovered in time-kill assay

| | CFU/mL recovered | | | |
|---|---|---|---|---|
| ATCC 33591 (MRSA) | T0 | T1 | T4 | T24 |
| Growth control | 8.5E+05 | 9.0E+05 | 3.0E+07 | 4.8E+09 |
| Neat Lot 00 | | 3.0E+05 | 2.9E+04 | 1.0E+01 |
| Neat Lot 01 | | 1.0E+05 | 3.0E+03 | 1.0E+01 |
| Neat Lot 02 | | 1.5E+05 | 4.2E+03 | 1.0E+01 |
| 50% Lot 00 | | 4.0E+05 | 5.7E+04 | 5.3E+02 |
| 50% Lot 01 | | 1.0E+05 | 3.1E+03 | 1.4E+03 |
| 50% Lot 02 | | 2.0E+05 | 2.1E+05 | 2.4E+04 |

Compared to the growth control (which contained no AB-101), both the undiluted AB-101 BRM and the 50% diluted AB-101 BRM reduced the bacterial density by 2 to 3 Log10 CFUs 4 hours post inoculation. By 24 hours, the MSSA and MRSA densities were reduced by ≥7 Log10 CFUs compared to growth control, and nearly all undiluted AB-101 test samples reduced the bacterial density to a level that is below the limit of detection for this assay. The time-kill assay therefore further confirmed the bactericidal effect of AB-101 against S. aureus.

### EXAMPLE 3

To demonstrate the effectiveness of AB-101 and how changes within different type of latex extract can change performance and well as showing that not all extracts, even in the same plant species, yields the same pharmaceutical grade performance, invitro assay of MSSA and MRSA were conducted. Measures included the minimum inhibitory concentration (MIC) which is the lowest concentration of AB-101 that prevents visible growth of the bacterium or pathogen, and minimum bactericidal concentration (MBC) which is the lowest concentration of an antibacterial agent required to kill a bacterium.

The comparison of AB-101 Lot 01 and 2 for MIC demonstrates a high effectiveness against MSSA and MRSA with particular emphasis on the Mupirocin resistant strain of MRSA. Mupirocin is a leading topical treatment for MRSA. Shown for the first time is the effectiveness of AB-101 against these pathogens and importantly an improvement over the leading current pharmaceutical treatment. Results are shown in Table 11.

TABLE 11

| | | MIC (% vol./vol.) | | | | MIC (μg/mL) | |
|---|---|---|---|---|---|---|---|
| Strain ID | Characteristic | AB-101 Lot 01 | | AB-101 Lot 02 | | Methicillin | Mupirocin |
| CDC 218 | MupirocinR, MRSA | 12.5 | 12.5 | 12.5 | 12.5 | >64 | >256 |
| CDC 224 | MupirocinR, MRSA | 12.5 | 12.5 | 12.5 | 12.5 | >64 | >256 |
| CDC 228 | MupirocinR, MRSA | 3.13 | 3.13 | 3.13 | 3.13 | 64 | >256 |
| 1674606 | MupirocinR, MSSA | 50 | 50 | 50 | 50 | 2-8 | >256 |
| 1674607 | MupirocinR, MRSA | 12.5 | 12.5 | 12.5 | 12.5 | >64 | >256 |
| 1674608 | MupirocinR, MSSA | 12.5 | 12.5 | 25 | 50 | 2-4 | >256 |
| 1674611 | MupirocinR, MRSA | 6.25 | 6.25 | 12.5 | 12.5 | 64 | >256 |
| CDC 480 | MupirocinS, MRSA | 12.5 | 25 | 12.5 | 25 | 32-64 | ≤0.25 |
| CDC 481 | MupirocinS, MRSA | 12.5 | 12.5 | 12.5 | 12.5 | 16-32 | ≤0.25 |
| CDC 482 | MupirocinS, MRSA | 12.5 | 12.5 | 12.5 | 12.5 | 8-32 | ≤0.25 |
| CDC 483 | MupirocinS, MRSA | 12.5 | 12.5 | 12.5 | 12.5 | 64 | ≤0.25 |
| CDC 220 | MupirocinS, MRSA | 12.5 | 12.5 | 6.25 | 12.5 | 64->64 | ≤0.25 |
| CDC 227 | MupirocinS, MRSA | 6.25 | 6.25 | 12.5 | 12.5 | 64 | ≤0.25-0.5 |
| CDC 461 | MupirocinS, MSSA | 12.5 | 12.5 | 12.5 | 12.5 | 4-8 | ≤0.25-1 |
| CDC 462 | MupirocinS, MSSA | 6.25 | 12.5 | 6.25 | 12.5 | 8 | ≤0.25 |
| CDC 463 | MupirocinS, MSSA | 6.25 | 6.25 | 12.5 | 12.5 | 8-16 | ≤0.25-0.5 |
| CDC 464 | MupirocinS, MSSA | 12.5 | 12.5 | 12.5 | 12.5 | 2 | ≤0.25 |
| CDC 484 | MupirocinS, MSSA | 12.5 | 12.5 | 12.5 | 12.5 | 2 | ≤0.25 |
| CDC 485 | MupirocinS, MSSA | 50 | 50 | 50 | 50 | 2-4 | ≤0.25 |
| ATCC 29213 | QC control | 12.5 | 50-12.5 | 12.5 | 12.5 | 1-2 | ≤0.25-0.5 |

Table 12 shows the comparison of AB-101 Lots 01 and 02 for MBC demonstrates a high effectiveness against MSSA and MRSA with particular emphasis on the mupirocin resistant strain of MRSA. Once again Mupirocin is a leading topical treatment for MRSA. Shown for the first time is the effectiveness of AB-101 against these pathogens and importantly an improvement over the leading current pharmaceutical treatment.

TABLE 12

| | | MBC (% vol./vol.) | | | |
|---|---|---|---|---|---|
| Strain ID | Characteristic | AB-101 Lot 1 | | AB-101 Lot 2 | |
| CDC 218 | MupirocinR, MRSA | 50 | 50 | 50 | 50 |
| CDC 224 | MupirocinR, MRSA | 50 | 50 | 50 | 50 |
| CDC 228 | MupirocinR, MRSA | 6.25 | 6.25 | 6.25 | 6.25 |
| 1674606 | MupirocinR, MSSA | >50 | >50 | >50 | >50 |
| 1674607 | MupirocinR, MRSA | 12.5 | 12.5 | 25 | 25 |
| 1674608 | MupirocinR, MSSA | 50 | 50 | 50 | >50 |
| 1674611 | MupirocinR, MRSA | 12.5 | 12.5 | 50 | 50 |
| CDC 480 | MupirocinS, MRSA | 50 | 50 | 50 | 50 |
| CDC 481 | MupirocinS, MRSA | 50 | 50 | 50 | 50 |
| CDC 482 | MupirocinS, MRSA | 50 | 50 | 50 | 50 |
| CDC 483 | MupirocinS, MRSA | 50 | 50 | 50 | 50 |
| CDC 220 | MupirocinS, MRSA | 25 | 25 | 25 | 25 |
| CDC 227 | MupirocinS, MRSA | 12.5 | 12.5 | 25 | 25 |
| CDC 461 | MupirocinS, MSSA | 50 | 50 | 50 | 50 |
| CDC 462 | MupirocinS, MSSA | 12.5 | 50 | 50 | 50 |
| CDC 463 | MupirocinS, MSSA | 12.5 | 12.5 | 12.5 | 12.5 |
| CDC 464 | MupirocinS, MSSA | >50 | 50 | 50 | 50 |
| CDC 484 | MupirocinS, MSSA | 50 | 50 | 50 | 50 |
| CDC 485 | MupirocinS, MSSA | 50 | 50 | 50 | 50 |
| ATCC 29213 | QC control | 50-12.5 | 50-12.5 | 50 | 50 |

Table 13 compares AB-101 lot X and Lot 00 for MIC because these lots have been shown elsewhere to have different composition. Lot X and Lot 00 are latex extracts of Croton lechleri Müll.Arg., the same species, grown in similar locations. Lot X demonstrates a significantly higher effectiveness against MSSA and MRSA. This data shows for the first time that not all latex extracts of Croton lechleri Müll.Arg. provide the same performance, even when the extracts are from the same species grown in similar locations.

TABLE 13

| | | MIC (% vol./vol.) | | | | MIC (μg/mL) | |
|---|---|---|---|---|---|---|---|
| Strain ID | Characteristic | AB-101 Lot 00 | | AB-101 Lot X | | Methicillin | Mupirocin |
| CDC 218 | MupirocinR, MRSA | 12.5 | 12.5 | 0.78 | 0.78 | >64 | >256 |
| CDC 224 | MupirocinR, MRSA | 12.5 | 12.5 | 0.78 | 0.78 | >64 | >256 |
| CDC 228 | MupirocinR, MRSA | 3.13 | 3.13 | 0.39 | 0.39 | 64 | >256 |
| 1674606 | MupirocinR, MSSA | >50 | >50 | 1.56 | 1.56 | 2-8 | >256 |
| 1674607 | MupirocinR, MRSA | 12.5 | 12.5 | 0.78 | 0.78 | >64 | >256 |
| 1674608 | MupirocinR, MSSA | 25 | 25 | 1.56 | 1.56 | 2-4 | >256 |
| 1674611 | MupirocinR, MRSA | 25 | 25 | 0.78 | 0.78 | 64 | >256 |
| CDC 480 | MupirocinS, MRSA | 12.5 | 12.5 | 1.56 | 1.56 | 32-64 | ≤0.25 |
| CDC 481 | MupirocinS, MRSA | 12.5 | 12.5 | 1.56 | 1.56 | 16-32 | ≤0.25 |
| CDC 482 | MupirocinS, MRSA | 12.5 | 12.5 | 0.78 | 0.78 | 8-32 | ≤0.25 |
| CDC 483 | MupirocinS, MRSA | 12.5 | 12.5 | 0.78 | 0.78 | 64 | ≤0.25 |
| CDC 220 | MupirocinS, MRSA | 12.5 | 12.5 | 0.78 | 0.78 | 64->64 | ≤0.25 |
| CDC 227 | MupirocinS, MRSA | 12.5 | 12.5 | 0.78 | 0.78 | 64 | ≤0.25-0.5 |
| CDC 461 | MupirocinS, MSSA | 50 | 50 | 0.78 | 0.78 | 4-8 | ≤0.25-1 |
| CDC 462 | MupirocinS, MSSA | 12.5 | 12.5 | 0.78 | 0.78 | 8 | ≤0.25 |
| CDC 463 | MupirocinS, MSSA | 12.5 | 12.5 | 1.56 | 0.78 | 8-16 | ≤0.25-0.5 |
| CDC 464 | MupirocinS, MSSA | >50 | >50 | 1.56 | 1.56 | 2 | ≤0.25 |
| CDC 484 | MupirocinS, MSSA | >50 | 25 | 0.78 | 1.56 | 2 | ≤0.25 |
| CDC 485 | MupirocinS, MSSA | >50 | >50 | 0.78 | 0.78 | 2-4 | ≤0.25 |
| ATCC 29213 | QC control | 12.5 | 12.5 | 1.56 | 1.56 | 1-2 | ≤0.25-0.5 |

Table 14 compares AB-101 lot X and Lot 00 for MBC because these lots have been shown elsewhere to have different composition. Lot X and Lot 00 are latex extracts of Croton lechleri Müll.Arg., the same species, grown in similar locations. Lot X demonstrates a significantly higher effectiveness against MSSA and MRSA. This data shows for the first time that not all latex extracts of Croton lechleri Müll.Arg. provide the same performance, even when the extracts are from the same species grown in similar locations.

TABLE 14

| | | MBC (% vol./vol.) | | | |
|---|---|---|---|---|---|
| Strain ID | Characteristic | AB-101 Lot 00 | | AB-101 Lot X | |
| CDC 218 | MupirocinR, MRSA | >50 | 50 | 0.78 | 0.78 |
| CDC 224 | MupirocinR, MRSA | >50 | >50 | 0.78 | 0.78 |
| CDC 228 | MupirocinR, MRSA | 12.5 | 12.5 | 0.39 | 0.78 |
| 1674606 | MupirocinR, MSSA | >50 | >50 | 1.56 | 1.56 |
| 1674607 | MupirocinR, MRSA | 25 | 25 | >50 | >50 |
| 1674608 | MupirocinR, MSSA | >50 | >50 | 1.56 | 1.56 |
| 1674611 | MupirocinR, MRSA | 50 | >50 | 0.78 | 0.78 |
| CDC 480 | MupirocinS, MRSA | >50 | >50 | 1.56 | 3.12 |
| CDC 481 | MupirocinS, MRSA | >50 | >50 | 1.56 | 1.56 |
| CDC 482 | MupirocinS, MRSA | >50 | >50 | 0.78 | 0.78 |
| CDC 483 | MupirocinS, MRSA | >50 | >50 | 0.78 | 0.78 |
| CDC 220 | MupirocinS, MRSA | 50 | 50 | 0.78 | 1.56 |
| CDC 227 | MupirocinS, MRSA | 25 | 25 | 1.56 | 0.78 |
| CDC 461 | MupirocinS, MSSA | >50 | >50 | 0.78 | 0.78 |
| CDC 462 | MupirocinS, MSSA | 50 | 50 | 0.78 | 0.78 |
| CDC 463 | MupirocinS, MSSA | 12.5 | 12.5 | 6.25 | 6.25 |
| CDC 464 | MupirocinS, MSSA | >50 | >50 | 1.56 | 1.56 |
| CDC 484 | MupirocinS, MSSA | >50 | >50 | 1.56 | 1.56 |
| CDC 485 | MupirocinS, MSSA | >50 | >50 | >50 | >50 |
| ATCC 29213 | QC control | 50 | 50 | 6.25 | 3.13 |

To demonstrate the effectiveness of AB-101 across other pathogens and in particular across gram negative (−) pathogens, Lot 01 and 02 were tested against Pseudomonas aeruginosa. All 20 of the Pseudomonas aeruginosa tested are resistant to multiple antibiotics, thereby they all fit the definition of being Multi-Drug Resistant (MDR). Of the 20, 5 are known to be Verona integron-encoded metallo-beta-lactamase (VIM)-producing Pseudomonas aeruginosa which are drug resistant strains of Pseudomonas aeruginosa, another 5 are know to be Klebsiella pneumoniae carbapenemase (KPC) producing Pseudomonas aeruginosa strains which are drug resistant strains of Pseudomonas aeruginosa, and 4 are known to be IMP-Type Metallo-β-Lactamase (IMP))-producing Pseudomonas aeruginosa which are drug resitant strains of Pseudomonas aeruginosa. The 6 remaining strains are known to be antibiotic resistant and are listed simply as MDR strains. Measures included the minimum inhibitory concentration (MIC) which is the lowest concentration of AB-101 that prevents visible growth of the bacterium or pathogen, and minimum bactericidal concentration (MBC) which is the lowest concentration of an antibacterial agent required to kill a bacterium.

Table 15 shows the comparison of AB-101 Lot 01 and 02 for MIC demonstrates a high effectiveness against Pseudomonas aeruginosa. Shown for the first time is the effectiveness of AB-101 against these pathogens and importantly an improvement over the leading current pharmaceutical treatment.

TABLE 15

| Pseudomonas | Pseudomonas | | | | | | | | |
|---|---|---|---|---|---|---|---|---|---|
| aeruginosa | aeruginosa | MIC (% AB-101)^{a} | | | | MIC (μg/mL)^{b} | | | |
| Strain ID | Characteristic | Lot 1 | | Lot 2 | | Imipenem | | Ciprofloxacin | |
| CDC 0230 | VIM | 25 | 25 | 25 | 25 | >64 | >64 | >64 | >64 |
| CDC 0239 | VIM | 12.5 | 12.5 | 12.5 | 12.5 | >64 | >64 | 32 | 32 |
| CDC 0254 | VIM | 25 | 25 | 25 | 25 | >64 | >64 | 32 | 32 |
| CDC 0255 | VIM | 25 | 25 | 25 | 25 | >64 | >64 | 32 | 32 |
| CDC 0509 | VIM | 25 | 25 | 25 | 25 | >64 | >64 | 32 | 32 |
| CDC 0231 | KPC | 25 | 25 | 25 | 25 | >64 | >64 | >64 | >64 |
| CDC 0356 | KPC | 25 | 25 | 25 | 25 | >64 | >64 | 0.125 | 0.125 |
| CDC 0441 | KPC | 25 | 25 | 25 | 25 | >64 | >64 | 2 | 2 |
| CDC 0516 | KPC | 25 | 25 | 25 | 25 | >64 | >64 | 0.125 | 0.125 |
| CDC 0518 | KPC | 12.5 | 12.5 | 12.5 | 12.5 | >64 | >64 | >64 | >64 |
| CDC 0092 | IMP | 12.5 | 12.5 | 12.5 | 50 | >64 | >64 | 32 | 32 |
| CDC 0103 | IMP | 12.5 | 12.5 | 12.5 | 12.5 | >64 | >64 | >32 | >32 |
| CDC 0439 | IMP | 12.5 | 12.5 | 12.5 | 12.5 | >64 | >64 | 32 | 32 |
| CDC 0241 | IMP | 12.5 | 12.5 | 12.5 | 12.5 | >64 | >64 | 16 | 16 |
| CDC 0508 | MDR | 12.5 | 12.5 | 12.5 | 12.5 | 16 | 16 | 2 | 2 |
| CDC 0353 | MDR | 25 | 25 | 12.5 | 12.5 | 16 | 16 | 16 | 16 |
| CDC 0357 | MDR | 25 | 25 | 25 | 25 | 16 | 16 | 32 | 32 |
| CDC 0246 | MDR | 12.5 | 12.5 | 12.5 | 12.5 | >64 | >64 | 32 | 32 |
| CDC 0250 | MDR | 25 | 12.5 | 12.5 | 25 | >64 | >64 | 32 | 32 |
| CDC 0064 | MDR | 25 | 25 | 25 | 25 | >64 | >64 | 16 | 16 |
| ATCC 27853 | QC strain | 25 | 25 | 25 | 25 | 2 | 2 | 0.25 | 0.25 |

Table 16 shows the comparison of Iminipenem and Cripofloxacin for MIC against quality control strain ATCC 27853.

TABLE 16

| Pseudomonas | CLSI QC Range | |
|---|---|---|
| aeruginosa | MIC (μg/ml) | |
| Strain | Imipenem | Ciprofloxacin |
| ATCC 27853 | 1-4 | 0.125-1 |

Table 17 shows the comparison of AB-101 Lots 01 and 02 for MBC demonstrates a high effectiveness against Pseudomonas aeruginosa. Shown for the first time is the effectiveness of AB-101 against these pathogens specifically for the multi-drug resistant pathogens.

TABLE 17

| Pseudomonas | | | | | | |
|---|---|---|---|---|---|---|
| aeruginosa | Pseudomonas | Pseudomonas | | | | |
| screen | aeruginosa | aeruginosa | MBC (% AB-101) | | | |
| number | ID | Characteristic | Lot 01 | | Lot 02 | |
| 1 | CDC 0230 | VIM | 25 | 25 | 25 | 25 |
| 2 | CDC 0239 | VIM | 25 | 25 | 25 | 25 |
| 3 | CDC 0254 | VIM | 25 | 25 | 25 | 25 |
| 4 | CDC 0255 | VIM | 25 | 25 | 25 | 25 |
| 5 | CDC 0509 | VIM | 25 | 25 | 25 | 25 |
| 6 | CDC 0231 | KPC | 25 | 25 | 25 | 25 |
| 7 | CDC 0356 | KPC | 25 | 25 | 25 | 25 |
| 8 | CDC 0441 | KPC | 25 | 25 | 25 | 25 |
| 9 | CDC 0516 | KPC | 25 | 25 | 25 | 25 |
| 10 | CDC 0518 | KPC | 25 | 25 | 25 | 25 |
| 11 | CDC 0092 | IMP | 25 | 25 | 25 | 50 |
| 12 | CDC 0103 | IMP | 25 | 25 | 25 | 25 |
| 13 | CDC 0439 | IMP | 25 | 25 | 25 | 25 |
| 14 | CDC 0241 | IMP | 25 | 12.5 | 25 | 25 |
| 15 | CDC 0508 | MDR | 50 | 50 | 25 | 25 |
| 16 | CDC 0353 | MDR | 25 | 25 | 25 | 25 |
| 17 | CDC 0357 | MDR | 25 | 25 | 25 | 25 |
| 18 | CDC 0246 | MDR | 50 | 12.5 | 25 | 50 |
| 19 | CDC 0250 | MDR | 25 | 25 | 25 | 25 |
| 20 | CDC 0064 | MDR | 25 | 25 | 25 | 25 |
| 21 | ATCC 27853 | QC strain | 25 | 25 | 25 | 25 |

AB-101 Lots 01, 02 and a purified extract of taspine for MIC are compared. The concentration of taspine at the highest concentration tested (i.e. 50%, relative to AB-101) is 10 μg/mL demonstrated for the first time from a bacteriologic perspective, taspine does not have activity as evaluated by this invitro test method against MSSA and MRSA. Taspine may have additional synergistic benefits to be included in the whole extract in the final product for topical treatment of ABSSSI. Results are shown in Table 18.

TABLE 18

| | | | | | | MIC (% | | | |
|---|---|---|---|---|---|---|---|---|---|
| | | | | | | relative | | | |
| | | | | | | to amount | | | |
| | | MIC (% vol./vol.) | | | | in AB-101) | | MIC (μg/mL) | |
| Strain ID | Characteristic | AB-101 Lot 01 | | AB-101 Lot 02 | | Taspine | | Methicillin | Mupirocin |
| CDC 218 | MupirocinR, MRSA | 12.5 | 12.5 | 12.5 | 12.5 | >50 | >50 | >64 | >256 |
| CDC 224 | MupirocinR, MRSA | 12.5 | 12.5 | 12.5 | 12.5 | >50 | >50 | >64 | >256 |
| CDC 228 | MupirocinR, MRSA | 3.13 | 3.13 | 3.13 | 3.13 | >50 | >50 | 64 | >256 |
| 1674606 | MupirocinR, MSSA | 50 | 50 | 50 | 50 | >50 | >50 | 2-8 | >256 |
| 1674607 | MupirocinR, MRSA | 12.5 | 12.5 | 12.5 | 12.5 | >50 | >50 | >64 | >256 |
| 1674608 | MupirocinR, MSSA | 12.5 | 12.5 | 25 | 50 | >50 | >50 | 2-4 | >256 |
| 1674611 | MupirocinR, MRSA | 6.25 | 6.25 | 12.5 | 12.5 | >50 | >50 | 64 | >256 |
| CDC 480 | MupirocinS, MRSA | 12.5 | 25 | 12.5 | 25 | >50 | >50 | 32-64 | ≤0.25 |
| CDC 481 | MupirocinS, MRSA | 12.5 | 12.5 | 12.5 | 12.5 | >50 | >50 | 16-32 | ≤0.25 |
| CDC 482 | MupirocinS, MRSA | 12.5 | 12.5 | 12.5 | 12.5 | >50 | >50 | 8-32 | ≤0.25 |
| CDC 483 | MupirocinS, MRSA | 12.5 | 12.5 | 12.5 | 12.5 | >50 | >50 | 64 | ≤0.25 |
| CDC 220 | MupirocinS, MRSA | 12.5 | 12.5 | 6.25 | 12.5 | >50 | >50 | 64->64 | ≤0.25 |
| CDC 227 | MupirocinS, MRSA | 6.25 | 6.25 | 12.5 | 12.5 | >50 | >50 | 64 | ≤0.25-0.5 |
| CDC 461 | MupirocinS, MSSA | 12.5 | 12.5 | 12.5 | 12.5 | >50 | >50 | 4-8 | ≤0.25-1 |
| CDC 462 | MupirocinS, MSSA | 6.25 | 12.5 | 6.25 | 12.5 | >50 | >50 | 8 | ≤0.25 |
| CDC 463 | MupirocinS, MSSA | 6.25 | 6.25 | 12.5 | 12.5 | >50 | >50 | 8-16 | ≤0.25-0.5 |
| CDC 464 | MupirocinS, MSSA | 12.5 | 12.5 | 12.5 | 12.5 | >50 | >50 | 2 | ≤0.25 |
| CDC 484 | MupirocinS, MSSA | 12.5 | 12.5 | 12.5 | 12.5 | >50 | >50 | 2 | ≤0.25 |
| CDC 485 | MupirocinS, MSSA | 50 | 50 | 50 | 50 | >50 | >50 | 2-4 | ≤0.25 |
| ATCC 29213 | QC control | 12.5 | 50-12.5 | 12.5 | 12.5 | >50 | >50 | 1-2 | ≤0.25-0.5 |

AB-101 Lots 01, 02 and a purified extract of taspine for MBC are compared. Demonstrated for the first time from a bacteriologic perspective, taspine does not have activity as evaluated by this invitro test method against MSSA and MRSA. Taspine may have additional synergistic benefits to be included in the whole extract in the final product for topical treatment of ABSSSI. Results are shown in Table 19.

TABLE 19

| | | | | | | MBC (% relative to | |
|---|---|---|---|---|---|---|---|
| | | MBC (% vol /vol.) | | | | amount in AB-101) | |
| Strain ID | Characteristic | AB-101 Lot 01 | | AB-101 Lot 02 | | Taspine | |
| CDC 218 | MupirocinR, MRSA | 50 | 50 | 50 | 50 | >50 | >50 |
| CDC 224 | MupirocinR, MRSA | 50 | 50 | 50 | 50 | >50 | >50 |
| CDC 228 | MupirocinR, MRSA | 6.25 | 6.25 | 6.25 | 6.25 | >50 | >50 |
| 1674606 | MupirocinR, MSSA | >50 | >50 | >50 | >50 | >50 | >50 |
| 1674607 | MupirocinR, MRSA | 12.5 | 12.5 | 25 | 25 | >50 | >50 |
| 1674608 | MupirocinR, MSSA | 50 | 50 | 50 | >50 | >50 | >50 |
| 1674611 | MupirocinR, MRSA | 12.5 | 12.5 | 50 | 50 | >50 | >50 |
| CDC 480 | MupirocinS, MRSA | 50 | 50 | 50 | 50 | >50 | >50 |
| CDC 481 | MupirocinS, MRSA | 50 | 50 | 50 | 50 | >50 | >50 |
| CDC 482 | MupirocinS, MRSA | 50 | 50 | 50 | 50 | >50 | >50 |
| CDC 483 | MupirocinS, MRSA | 50 | 50 | 50 | 50 | >50 | >50 |
| CDC 220 | MupirocinS, MRSA | 25 | 25 | 25 | 25 | >50 | >50 |
| CDC 227 | MupirocinS, MRSA | 12.5 | 12.5 | 25 | 25 | >50 | >50 |
| CDC 461 | MupirocinS, MSSA | 50 | 50 | 50 | 50 | >50 | >50 |
| CDC 462 | MupirocinS, MSSA | 12.5 | 50 | 50 | 50 | >50 | >50 |
| CDC 463 | MupirocinS, MSSA | 12.5 | 12.5 | 12.5 | 12.5 | >50 | >50 |
| CDC 464 | MupirocinS, MSSA | >50 | 50 | 50 | 50 | >50 | >50 |
| CDC 484 | MupirocinS, MSSA | 50 | 50 | 50 | 50 | >50 | >50 |
| CDC 485 | MupirocinS, MSSA | 50 | 50 | 50 | 50 | >50 | >50 |
| ATCC 29213 | QC control | 50-12.5 | 50-12.5 | 50 | 50 | >50 | >50 |

### EXAMPLE 4

Ten patients with 18 MRSA drug resistant skin infections (1 pressure ulcer and 17 lesions defined as cuts and scrapes secondarily infected with MRSA) have been treated with AB-101. Current treatments for drug resistant skin infections include topical, oral, intravenous (IV) drugs, and combinations of these when single drug therapy fails. Each of these treatments exhibits problems beginning with failure to cure the infection (i.e. bacterial resistance to the drug), hospitalization, expensive combination drug therapies, risk of death, whole body drug exposure (potentiating further bacterial drug resistance), multiple varied side effects and slow wound healing.

All MRSA infections were cultured and verified by the patient's physicians.

Patient 1, an elderly female patient in an extended care facility was diagnosed with a chronic Mupirocin-resistant MRSA-infected ulcer, and was switched from Mupirocin to AB-101 6% ointment delivery. The staff was instructed to apply the ointment twice daily to the ulcer, and measure the ulcer size 3 times a week. Over a period of 4 weeks the measurement of the lesion size decreased to the point where the ulcer was almost completely healed and the lesion was not measurable.

Patient 2, an adult female subject was exposed to MRSA through another infected individual from a wrestling tournament and diagnosed with 3 infected lesions. She signed a consent form and was given a 1 ounce bottle of AB-101 (undiluted liquid; 100% concentration) and written instructions to apply a drop to her lesion 3 times daily and rub it gently over the lesion until dry. Nineteen days later her 3 lesions were healed and the MRSA infection eradicated.

Patient 3, an adult male subject was exposed to MRSA through another infected individual from a wrestling tournament and diagnosed with 3 infected lesions. He signed a consent form and was given a 1 ounce bottle of AB-101 (undiluted liquid; 100% concentration) and written instructions to apply a drop to his lesion 3 times daily and rub it gently over the lesion until dry. Twelve days later his 3 lesions were healed and the MRSA infection eradicated.

Patient 4, a teenage male subject was exposed to MRSA through another infected individual from a wrestling tournament and diagnosed with 5 infected lesions. He and his parent signed a consent form and was given a 1 ounce bottle of AB-101 (undiluted liquid; 100% concentration) and written instructions to apply a drop to his lesion 3 times daily and rub it gently over the lesion until dry. Twenty days later his 5 lesions were healed and the MRSA infection eradicated.

Six additional teenage male patients, patients 5 through 10, were diagnosed with 1 MRSA infected lesion each. All patients (or their parent or guardian if the patient was a minor) signed a consent form and were given a 1-ounce bottle of AB-101 (undiluted liquid, 100% concentration) with written instructions to apply 1 drop to each lesion 3 times daily and rub it gently over the lesion until dry. Patients 5-10's lesions were healed and the MRSA infections eradicated within 21 days.

No patient reported drug resistance, any side effects, pain, scarring, slow wound healing, hospitalization or combination therapy as a result of AB-101 use. Use of AB-101 resulted in no reported or observed irritation, hypersensitivity or photosensitivity.

Table 20 presents AB-101 human primary and secondary outcomes and final assessments in MRSA drug resistant skin infections for the 10 patients in this report.

TABLE 20

| | | | Primary | | |
|---|---|---|---|---|---|
| Patient | | | Outcome/ | | Final |
| Demographics | Diagnosis | Treatment | Response | Secondary Outcomes | Assessment |
| Patient 1: | 1 MRSA | MRSA-1 | Lesion | Did not report: whole body drug exposure, | Complete |
| Elderly female | Pressure ulcer | Ointment: 3 × | eradicated | drug resistance, side effects, pain, scarring, | Response |
| | Bactroban ® | Daily to | After 4 weeks | slow wound healing, hospitalization, | |
| | Resistant | affected area | of treatment | combination drug treatment | |
| Patient 2: | 3 MRSA | MRSA-1 | All lesions | Did not report: whole body drug exposure, | Complete |
| Adult female | lesions | Liquid: 3 × | eradicated | drug resistance, side effects, pain, scarring, | Response |
| | | Daily to | After 19 days | slow wound healing, hospitalization, | |
| | | affected area | of treatment | combination drug treatment | |
| Patient 3: | 3 MRSA | MRSA-1 | All lesions | Did not report: whole body drug exposure, | Complete |
| Adult male | lesions | Liquid: 3 × | eradicated | drug resistance, side effects, pain, scarring, | Response |
| | | Daily to | After 12 days | slow wound healing, hospitalization, | |
| | | affected area | of treatment | combination drug treatment | |
| Patient 4: | 5 MRSA | MRSA-1 | All lesions | Did not report: whole body drug exposure, | Complete |
| Teenage male | lesions | Liquid: 3 × | eradicated | drug resistance, side effects, pain, scarring, | Response |
| | | Daily to | After 20 days | slow wound healing, hospitalization, | |
| | | affected area | of treatment | combination drug treatment | |
| Patient 5: | 1 MRSA | MRSA-1 | All lesion(s) | Did not report: whole body drug exposure, | Complete |
| Teenage male | lesion | Liquid: 3 × | eradicated | drug resistance, side effects, pain, scarring, | Response |
| | | Daily to | After 21 days | slow wound healing, hospitalization, | |
| | | affected area | of treatment | combination drug treatment | |
| Patient 6: | 1 MRSA | MRSA-1 | All lesion(s) | Did not report: whole body drug exposure, | Complete |
| Teenage male | lesion | Liquid: 3 × | eradicated | drug resistance, side effects, pain, scarring, | Response |
| | | Daily to | After 21 days | slow wound healing, hospitalization, | |
| | | affected area | of treatment | combination drug treatment | |
| Patient 7: | 1 MRSA | MRSA-1 | All lesion(s) | Did not report: whole body drug exposure, | Complete |
| Teenage male | lesion | Liquid: 3 × | eradicated | drug resistance, side effects, pain, scarring, | Response |
| | | Daily to | After 21 days | slow wound healing, hospitalization, | |
| | | affected area | of treatment | combination drug treatment | |
| Patient 8: | At least 1 | MRSA-1 | All lesion(s) | Did not report: whole body drug exposure, | Complete |
| Teenage male | MRSA | Liquid: 3 × | eradicated | drug resistance, side effects, pain, scarring, | Response |
| | lesion | Daily to | After 21 days | slow wound healing, hospitalization, | |
| | | affected area | of treatment | combination drug treatment | |
| Patient 9: | At least 1 | MESA-1 | All lesion(s) | Did not report: whole body drug exposure, | Complete |
| Teenage male | MRSA | Liquid: 3 × | eradicated | drug resistance, side effects, pain, scarring, | Response |
| | lesion | Daily to | After 21 days | slow wound healing, hospitalization, | |
| | | affected area | of treatment | combination drug treatment | |
| Patient 10: | At least 1 | MRSA-1 | All lesion(s) | Did not report: whole body drug exposure, | Complete |
| Teenage male | MRSA | Liquid: 3 × | eradicated | drug resistance, side effects, pain, scarring, | Response |
| | lesion | Daily to | After 21 days | slow wound healing, hospitalization, | |
| | | affected area | of treatment | combination drug treatment | |

AB-101 human results for irritation, hypersensitivity and photosensitivity in MRSA drug resistant skin infections for the 10 patients of this report are presented in Table 21.

TABLE 21

| Patient | | | |
|---|---|---|---|
| Demographics | Irritation | Hypersensitivity | Photosensitivity |
| Patient 1: | No irritation | No hypersensitivity | No photosensitivity |
| Elderly female | observed or reported | observed or reported | observed or reported |
| Patient 2: | No irritation | No hypersensitivity | No photosensitivity |
| Adult female | observed or reported | observed or reported | observed or reported |
| Patient 3: | No irritation | No hypersensitivity | No photosensitivity |
| Adult male | observed or reported | observed or reported | observed or reported |
| Patient 4: | No irritation | No hypersensitivity | No photosensitivity |
| Teenage male | observed or reported | observed or reported | observed or reported |
| Patient 5: | No irritation | No hypersensitivity | No photosensitivity |
| Teenage male | observed or reported | observed or reported | observed or reported |
| Patient 6: | No irritation | No hypersensitivity | No photosensitivity |
| Teenage male | observed or reported | observed or reported | observed or reported |
| Patient 7: | No irritation | No hypersensitivity | No photosensitivity |
| Teenage male | observed or reported | observed or reported | observed or reported |
| Patient 8: | No irritation | No hypersensitivity | No photosensitivity |
| Teenage male | observed or reported | observed or reported | observed or reported |
| Patient 9: | No irritation | No hypersensitivity | No photosensitivity |
| Teenage male | observed or reported | observed or reported | observed or reported |
| Patient 10: | No irritation | No hypersensitivity | No photosensitivity |
| Teenage male | observed or reported | observed or reported | observed or reported |

The 10 patients reported here demonstrated the efficacy for AB-101 in treating MRSA drug resistant skin infections and that use of AB-101 eliminated the significant problems of current treatments for MRSA drug resistant skin infections.

### EXAMPLE 5

### Prophetic

Objective: The objective of this clinical study is to determine whether topical application of AB-101 (a pharmaceutical composition of the present invention) is safe and effective in the treatment of Acute Bacterial Skin or Skin Structure Infections (ABSSSI).

Design: Up to 400 participants with an ABSSSI be enrolled and directed to self-administer or have administered for them the study product to the infection site one times per day or two times per day (in the morning and at bedtime) or three times per day or four times per day, after washing the infected area. The application course will be for a period of up to 4 weeks. Participants will be evaluated on Day 3, 7, 10, 14, 21 and 28.

Study Population: Male and female participants aged 18 and older with a clinical diagnosis of an ABSSSI with no other meaningful uncontrolled systemic diseases, non-pregnant, non-lactating, practicing acceptable contraception, can participate in this study.

Rational: Methicillin-resistant Staphylococcus aureus (MRSA) infection is caused by a type of staphylococcus bacteria that's become resistant to many of the antibiotics used to treat ordinary staphylococcus infections. Most MRSA infections occur in people who've been in hospitals or other health care settings, such as nursing homes and dialysis centers. When it occurs in these settings, it's known as health care-associated MRSA (HA-MRSA). HA-MRSA infections typically are associated with invasive procedures or devices, such as surgeries, intravenous tubing or artificial joints. Another type of MRSA infection has occurred in the wider community—among healthy people. This form, community-associated MRSA (CA-MRSA), often begins as a painful skin boil. It's spread by skin-to-skin contact. At-risk populations include groups such as high school wrestlers, child care workers and people who live in crowded conditions.

Staphylococcus skin infections, including MRSA, generally start as swollen, painful red bumps that might resemble pimples or spider bites. The affected area might be:
Warm to the touch
Full of pus or other drainage
Accompanied by a fever

These can quickly turn into deep, painful abscesses that require surgical draining. Sometimes the bacteria remain confined to the skin. But they can also burrow deep into the body, causing potentially life-threatening infections in bones, joints, surgical wounds, the bloodstream, heart valves and lungs.

Bacteriology analysis of AB-101 has demonstrated that it has antibacterial properties that may be effective in the treatment of ABSSSIs. Based on these data, Alphyn Biologics intends to examine the safety and effectiveness of AB-101 in the treatment of ABSSSIs.

Study Design. This will either be an open label, single-arm study or a blinded randomized controlled two or three arm study to evaluate the safety and effectiveness of AB-101 in participants with lesions related to MRSA. Up to 400 participants with a clinical diagnosis of MRSA will be enrolled and directed to self-administer or have administered for them AB-101 to each qualifying lesion 1, 2, 3, or 4 times per day. The application course will be for a period of up to 4 weeks. Participants will be evaluated on Day 3, 7, 10, 14, 21 and 28. Once the Investigator has determined that a participant meets the eligibility criteria, the next sequential participant enrollment number will be assigned. Participants who discontinue for non-compliance or withdrawal of consent after receiving study product who have not been treated for a minimum of 1 week will be replaced; participants who fail the screening process and do not receive any study product will be replaced.

Dosing Schedule and Instructions. AB-101 will be supplied in a tubes of 22 grams or 15 grams or similar. Participants will be provided with sufficient study product to last through out the application period (up to 2 weeks). If all lesions are cleared, participant should still come for their next scheduled study visit, which will be their end of study visit.

AB-101 should be applied to each qualifying lesion once, twice, thrice or four times per day. Apply one small ribbon of AB-101 to the lesion.

Participant or their caregiver will complete a Participant Diary to document product application.

Criteria for Disease Evaluation. At Day 0, an initial disease evaluation will be performed. Disease evaluation will be performed and recorded at each visit through Day 28. At each visit the following will be assessed:
Overall response assessment: complete, partial, or no resolution of lesion present at baseline (see Table 22 for scoring)
Bacteriology

TABLE 22
Overall Response Assessment

| GRADE | CRITERIA |
|---|---|
| 1 | Complete resolution of lesion present at baseline |
| 2 | Partial resolution |
| 3 | No resolution of lesions present at baseline |

1. A method of treating an acute bacterial skin or skin structure infection in a subject in need thereof comprising topically administering a therapeutically effective amount of a pharmaceutical composition containing filtered latex of Croton lechleri, wherein the Croton lechleri contains at least about 110 PPM of Gallocatechin, at least about 780 PPM of Epigallocatechin, about at least about 1.6 PPM of Catechin at least about 2 PPM of Epicatechin, at least about 45 PPM Taspine.

2. The method of claim 1, wherein the Croton lechleri is Croton lechleri Müll.Arg.

3. The method of claim 1, wherein the acute bacterial skin or skin structure infection is selected from the group consisting of Streptococcus pyogenes infection, Staphylococcus aureus infection, methicillin-resistant Staphylococcus aureus (MRSA) infection, Mupirocin-resistant MRSA infection, Enterococcus faecalis infection, Gram-positive bacteria infection, Gram-negative bacteria infection, cellulitis/erysipelas, wound infection, burn infection, major cutaneous abscesses, impetigo, Mupirocin-resistant impetigo, Vancomycin resistant bacteria infection, Mupirocin resistant bacteria infection, Clostridium difficile infection, drug-resistant Neisseria gonorrhoeae infection, Streptococcus pneumoniae infection, drug-resistant Streptococcus pneumoniae infection, drug-resistant Klebsiella pneumoniae infection, drug-resistant Malaria infection, Multi-drug resistant (MDR) infection, Extensively drug-resistant (XDR) Tuberculosis infection, Escherichia coli (E. coli) infection, Shiga toxin-producing Escherichia coli (E. coli) infection, infections caused by bacteria possessing Enzyme NDM-1 (New Delhi Metallo-beta-lactamase-1), Clostridium difficile infection, Enterococcus infection, Mycobacterium tuberculosis infection, Mycoplasma genitalium infection, Streptococcus infection, Campylobacter infection, Neisseria gonorrhoeae infection, Gamma proteobacteria infection, Enterobacteriaceae infection, Carbapenem-Resistant Enterobacteriaceae, infection, Klebsiella pneumoniae infection, Salmonella infection, E. coli infection, Pseudomonadales infection, Acinetobacter infection, Pseudomonas aeruginosa infection, MDR Pseudomonas aeruginosa infection, and Coagulase-negative Staphylococcus infection.

4. The method of claim 3, wherein the acute bacterial skin or skin structure infection is selected from the group consisting of Streptococcus pyogenes infection, Staphylococcus aureus infection, methicillin-resistant Staphylococcus aureus (MRSA) infection, Mupirocin-resistant MRSA infection, Enterococcus faecalis infection, Streptococcus pneumoniae infection, Escherichia coli (E. coli) infection, Pseudomonas aeruginosa infection, MDR Pseudomonas aeruginosa infection, and Coagulase-negative Staphylococcus infection.

5. The method of claim 4, wherein the acute bacterial skin or skin structure infection is methicillin-resistant Staphylococcus aureus infection.

6. The method of claim 4, wherein the acute bacterial skin or skin structure infection is Mupirocin-resistant MRSA infection.

7. The method of claim 4, wherein the acute bacterial skin or skin structure infection is Pseudomonas aeruginosa infection.

8. The method of claim 4, wherein the acute bacterial skin or skin structure infection is MDR Pseudomonas aeruginosa infection.

9. The method of claim 1, wherein the pharmaceutical composition is a liquid, ointment, lotion, or cream.

10. The method of claim 1 wherein the administration is until the acute bacterial skin or skin structure infection is treated.

11. The method of claim 1, wherein the pharmaceutical composition is topically administered directly to the acute bacterial skin or skin structure infection.

12. The method of claim 1, wherein the pharmaceutical composition is topically administered directly to the lesion that results from an acute bacterial skin or skin structure infection.

13. The method of claim 1, wherein the treatment lasts until the acute bacterial skin or skin structure infection is treated.

14. The method of claim 1, wherein the pharmaceutical composition further comprises a pharmaceutically acceptable carrier.

15. A pharmaceutical composition comprising a therapeutically effective amount of filtered latex of Croton lechleri Müll.Arg, wherein the Croton lechleri Müll.Arg contains at least about 110 PPM of Gallocatechin, at least about 780 PPM of Epigallocatechin, at least about 1.6 PPM of Catechin, at least about 1.6 PPM of Epicatechin, at least about 45 PPM Taspine and wherein the pharmaceutical composition is suitable for topical administration.

16. The pharmaceutical composition of claim 14, wherein the pharmaceutical composition is a liquid, ointment, lotion, or cream.

17. The pharmaceutical composition of claim 14, wherein the therapeutically effective amount of filtered latex of Croton lechleri Müll.Arg is about 3 to 100 wt %.

18. The pharmaceutical composition of claim 14, wherein the pharmaceutical composition further comprises a pharmaceutically acceptable carrier.

19. The pharmaceutical composition of claim 14, wherein the pharmaceutical composition has a MIC of at least 50% concentration of AB-101.
